(19) United States
(12) Patent Application Publication
(10) Pub. No.: US 2005/0075358 A1
(43) Pub. Date: Apr. 7, 2005

(54) Methods for treating IGF1R-inhibitor induced hyperglycemia

(76) Inventors: Joan M. Carboni, Yardley, PA (US); Ricardo M. Attar, Lawrenceville, NJ (US); Marco M. Gottardis, Princeton, NJ (US); Jean M. Whaley, Bedminster, NJ (US); Thomas W. Harrity, Middlesex, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/958,869

(22) Filed: Oct. 5, 2004

Related U.S. Application Data
(60) Provisional application No. 60/508,890, filed on Oct. 6, 2003.

(51) Int. Cl. A61K 31/513; A61K 31/4439; A61K 31/426; A61K 31/4184; A61K 31/155

(52) U.S. Cl. 514/269; 514/338; 514/394; 514/342; 514/369; 514/636

(57) ABSTRACT

Methods for treating cancer using IGF1R inhibitors in combination with insulin sensitizers are provided for the treatment or prevention of hyperglycemia.

### RELATED APPLICATIONS

This application claims priority benefit under Title 35 § 119(e) of U.S. provisional Application No. 60/508,890, filed Oct. 6, 2003, the contents of which are herein incorporated by reference.

### BACKGROUND

The present invention relates to methods for treating cancer using tyrosine kinase inhibitors. Cancer is a disease that is characterized by an overexpression or upregulation of tyrosine kinase activity. Tyrosine kinases play a critical role in signal transduction for several cellular functions including cell proliferation, carcinogenesis, apoptosis, and cell differentiation (Plowman, G. D.; Ullrich, A.; Shawver, L. K.: Receptor Tyrosine Kinases As Targets For Drug Intervention. DN&P (1994) 7: 334-339).

Epidemiologic evidence suggests that the overexpression or activation of receptor protein tyrosine kinases leading to constitutive mitogenic signaling is an important factor in a growing number of human malignancies. One tyrosine kinase that has been implicated in these processes is the IGF1 receptor (IGF1R). Several clinical reports stress the important role of the IGF-1 pathway in human tumor development. IGF1R overexpression is frequently found in various tumors, such as breast, colon, lung, sarcoma, and is often associated with an aggressive phenotype. High circulating IGF1 concentrations are strongly correlated with prostate, lung and breast cancer risk.

It has been found that inhibition of the IGF1 receptor is useful for cancer treatment. Several in vitro and in vivo strategies have provided the proof of principal that inhibition of IGF1R signaling reverses the transformed phenotype and inhibits tumor cell growth. However, although IGF1R inhibitors are promising chemotherapeutic agents, use of these drugs presents a challenge because of the homology between the IGF1 receptor and the insulin receptor (IR). The insulin receptor kinase domain is 84% homologous to the IGF1R kinase domain and there are few reports of IGF1R tyrosine kinase inhibitors that are selective over the insulin receptor.

The insulin receptor is a protein expressed on the surface of cells, especially muscle, fat and liver cells. When insulin binds to the insulin receptor it initiates an insulin-signaling pathway within the cell that results in a variety of physiological reactions, including the movement of glucose into the cell. An interruption of this cell-signaling pathway can lead to a variety of conditions, including hyperglycemia.

It has been found that administration of small molecule IGF1R inhibitors induces hyperglycemia, presumably through a block of the insulin receptor. See, WO 02/102804. Accordingly, there is a need in the art to find methods to treat or prevent IGF1R inhibitor-induced hyperglycemia so that the full potential of these promising new drugs can be realized.

Insulin and insulin secretatogues are not viable options in treating IGF1R-induced hyperglycemia because their primary mechanisms of action involve increased levels of insulin and presumably require intact insulin receptors.

Insulin sensitizers, such as metformin, are widely used drugs that are used to treat Type II diabetes or non-insulin dependent diabetes. The mechanism of action of metformin has not been well elucidated. However, the scientific literature suggests that that clinical efficacy requires the presence of insulin. See, Weinsperger and Bailey, Drugs 58: Supp. 1, 31-39 (1999) and Bailer, Diabetes Care, 15: 755 to 772 (1992). Metformin is known to inhibit production of glucose from the liver and some studies suggest that this action involves insulin receptor (IR) activation. See, Gunton et al. J. of Clin. Endocrinology and Metabolism, 88(3): 1323-1332. In support of this view, it has been found that treatment of individuals having known IR mutations with an insulin-sensitizer such as metformin is not very effective. See, Vestegaard et al., J. Internal Med. 250: 406-414 (2001). Gunton et al. also report that metformin fails to increase IR phosphorylation when given in combination with an IR inhibitor, suggesting that inhibition of the IR receptor caused by an IGF1R inhibitor would also block the ability of an insulin-sensitizer to reduce or prevent IGF1R-induced hyperglycemia.

Currently, there is a need in the art for cancer treatments with IGF1R inhibitors that do not result in hyperglycemia.

### SUMMARY

Surprisingly, insulin-sensitizers have been found to be effective for treating, reducing or preventing IGF1R inhibitor-induced hyperglycemia. Accordingly, the present invention is directed to methods of treating cancer, comprising administering to a mammal in need of such treatment an effective amount of an IGF1R inhibitor in combination with an effective amount of an insulin-sensitizer to reduce, treat, or prevent hyperglycemia.

### BRIEF DESCRIPTION OF THE FIGURES

FIG. 1: Illustrates the effects of an IGF1R inhibitor on the plasma glucose levels in a rat when treated with an IGF1R inhibitor.

FIG. 2: Illustrates that IGF1R induced hyperglycemia is preventable in rats being treated with IGF1R inhibitors.

### DETAILED DESCRIPTION OF THE INVENTION

According to the present invention, methods are provided for treating or preventing IGF1R inhibitor-induced hyperglycemia in a patient undergoing treatment with an IGF1R inhibitor. The discovery that an insulin-sensitizer has the ability to treat or prevent IGF1R induced hyperglycemia without altering the cellular proliferation effects or antitumor efficacy of these inhibitors will enhance drug discovery efforts in this area.

As used herein, hyperglycemia is a defined as a measure of fasting glucose levels that are above 110 mg/dl and postprandial glucose levels that are above 140 mg/dl.

“Acceptable glucose levels” are those that are below about 140 mg/dl postprandially, and below about 110 mg/dl in the fasting state.

As used herein, the phrase “IGF1R-induced hyperglycemia” refers to a state of hyperglycemia that is caused by administration of an IGF1R inhibitor to a mammal in need of treatment for a disease that is susceptible to IGF1R inhibition, such as cancer.

The term “alkyl” herein alone or as part of another group refers to a monovalent alkane (hydrocarbon) derived radical containing from 1 to 12 carbon atoms unless otherwise defined. An alkyl group is an optionally substituted straight, branched or cyclic saturated hydrocarbon group. When substituted, alkyl groups may be substituted with up to four substituent groups, R as defined, at any available point of attachment. When the alkyl group is said to be substituted with an alkyl group, this is used interchangeably with “branched alkyl group”. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the like. Exemplary substituents may include but are not limited to one or more of the following groups: hydroxy, halo (such as F, Cl, Br, I), haloalkyl (such as CCl₃ or CF₃), alkoxy, alkylthio, cyano, carboxy (—COOH), alkylcarbonyl (—C(O)R), alkoxycarbonyl (—OCOR), amino, carbamoyl (—NHCOOR or —OCONHR), urea (—NHCONHR), thiol, (—SH), sulfoxy, sulfonyl, aryl, heteroaryl, and heterocycloalkyl. Alkyl groups as defined may also comprise one or more carbon to carbon double bonds or one or more carbon to carbon triple bonds. Alkyl groups may also be represented by the formula alkyl-R²⁵. In preferred embodiments, the alkyl group is a methyl, ethyl, propyl or butyl group and include substituted methyl, ethyl, propyl or butyl groups.

The term “alkenyl” herein alone or as part of another group refers to a hydrocarbon radical straight, branched or cyclic containing from 2 to 12 carbon atoms and at least one carbon to carbon double bond. An alkenyl group may be optionally substituted in the same manner as described for an alkyl group.

The term “alkynyl” herein alone or as part of another group refers to a hydrocarbon radical straight, branched or cyclic containing from 2 to 12 carbon atoms and at least one carbon to carbon triple bond. An alkynyl group may be optionally substituted in the same manner as described for an alkyl group.

The term “alkoxy” as used alone or in combination herein refers to a straight or branched chain alkyl group covalently bonded to the parent molecule through an oxygen atom linkage containing from one to ten carbon atoms and the terms “C₁₋₆ alkoxy” and “lower alkoxy” refer to such groups containing from one to six carbon atoms, examples include, but are not limited to, methoxy, ethoxy, propoxy, isopropoxy, butoxy, t-butoxy and the like. The term “optionally substituted” when used in connection with an alkoxy substituent refers to the replacement of up to two hydrogens, preferably on different carbon atoms with a radical selected form the group of lower alkyl, phenyl, cyano, halo, trifluoromethyl, nitro, hydroxy, alkanoyl, amino, monoalkyl amino and dialkylamino. Alkoxy groups may be substituted in the same manner that alkyl groups can be substituted as described above.

The term “sulfoxy” herein alone or as part of a group refers to —SO and may be substituted with, for example, alkyl or aryl groups.

The term “sulfonyl” herein alone or as part of a group refers to —SO₂ and may be substituted with alkyl or aryl groups.

The term “amino” herein alone or as part of another group refers to —NH₂. An “amino” may optionally be substituted with one or two substituents, which may be the same or different, such as alkyl, aryl, arylalkyl, alkenyl, alkynyl, heteroaryl, heteroarylalkyl, cycloheteroalkyl, cycloheteroalkylalkyl, cycloalkyl, cycloalkylalkyl, haloalkyl, hydroxyalkyl, alkoxyalkyl or thioalkyl. Preferred substituents include alkylamino and dialkylamino, such as methylamino, ethylamino, dimethylamino, and diethylamino. These substituents may be further substituted with a carboxylic acid or any of the alkyl or aryl substituents set out herein. In addition, the amino substituents may be taken together with the nitrogen atom to which they are attached to form 1-pyrrolidinyl, 1-piperidinyl, 1-azepinyl, 4-morpholinyl, 4-thiamorpholinyl, 4-sulfoxymorpholine, 4-sulfonylmorpholine, 1-piperazinyl, 4-alkyl-1-piperazinyl, 4-arylalkyl-1-piperazinyl, 4-diarylalkyl-1-piperazinyl, 1-homopiperazinyl, 4-alkyl-1-homopiperazinyl, 4-arylalkyl-1-homopiperazinyl, 4-diarylalkyl-1-homopiperazinyl; 1-pyrrolidinyl, 1-piperidinyl, or 1-azepinyl, optionally substituted with alkyl, alkoxy, alkylthio, halo, trifluoromethyl or hydroxy.

The term “aryl” herein alone or as part of another group refers to monocyclic or bicyclic aromatic rings, e.g. phenyl, substituted phenyl and the like, as well as groups which are fused, e.g., napthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Aryl groups may optionally be substituted with one or more groups including, but not limited to halogen, alkyl, alkenyl, alkynyl, alkoxy, hydroxy, carboxy, carbamoyl, alkyloxycarbonyl, alkylaminocarbonyl, nitro, trifluoromethyl, amino, cycloalkyl, cyano, alkyl S(O)ₘ (m=O, 1, 2), or thiol. Aryl groups may also be substituted with heterocycloalkyl and heterocycloaryl groups to form fused rings, such as dihydrobenzfuranyl, oxindolyl, indolyl, indolinyl, oxindolyl, benzoxazolidinonyl, benzoxazolinyl and benzoxazolidinone.

The term “cycloalkyl” herein alone or as part of another group refers to fully saturated and partially unsaturated hydrocarbon rings of 3 to 9, preferably 3 to 7 carbon atoms. Further, a cycloalkyl may be substituted. A substituted cycloalkyl refers to such rings having one, two, or three substituents, preferably one, selected from the group consisting of halo, alkyl, substituted alkyl, alkenyl, alkynyl, nitro, cyano, oxo (═O), hydroxy, alkoxy, thioalkyl, —CO₂H, —OC(═O)H, CO₂-alkyl, —OC(═O)alkyl, ═N—OH, ═N—O-alkyl, aryl, heteroaryl, heterocyclo, a five or six membered ketal (i.e. 1,3-dioxolane or 1,3-dioxane), —NR′R″, —C(═O)NR′R″, —OC(═O)NR′R″, —NR′CO₂′R″, —NRC(═O)R″, —SO₂NR′R″, and —NR′ SO₂′R″, wherein each of R′ and R″ is independently selected from hydrogen, alkyl, substituted alkyl, and cycloalkyl, or R′ and R″ together form a heterocyclo or heteroaryl ring. Cycloalkyl groups may also be substituted with hetero atoms such as O, N, and S to form heterocycloalkyl groups. Preferred heterocycloalkyl groups include optionally substituted morpholine, homomorpholine (7 membered ring), thiomorpholine, piperazine, homopiperazine (7 membered ring), and piperidine, wherein said substituents may include further heterocycloalkyl groups that may also be further substituted.

The term “heteroaryl” herein alone or as part of another group refers to substituted and unsubstituted aromatic 5 or 6 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain zero, one, two or three substituents selected from the group consisting of halo, alkyl, substituted alkyl, alkenyl, alkynyl, nitro, cyano, hydroxy, alkoxy, thioalkyl, —CO₂H, —OC(═O)H, —CO₂-alkyl, —OC(═O)alkyl, phenyl, benzyl, phenylethyl, phenyloxy, phenylthio, cycloalkyl, substituted cycloalkyl, heterocyclo, heteroaryl, —NR′R″, —C(═O)NR′R″, —OC(═O)NR′R″, —NR′CO₂′R″, —NRC(═O)R″, —SO₂NR′R″, and —NR′ SO₂R″, wherein each of R′ and R″ is independently selected from hydrogen, alkyl, substituted alkyl, and cycloalkyl, or R′ and R″ together form a heterocyclo or heteroaryl ring.

Exemplary monocyclic heteroaryl groups include pyrrolyl, pyrrolidinyl, imidazolinyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl, and the like.

Exemplary bicyclic heteroaryl groups include indolyl, indolinyl, oxindolyl, benzoxazolidinone, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like.

Exemplary tricyclic heteroaryl groups include carbazolyl, benzindolyl, phenanthrollinyl, acridinyl, phenanthridinyl, xanthenyl and the like.

The term “halogen” or “halo” herein alone or as part of another group refers to chlorine, bromine, fluorine or iodine selected on an independent basis.

The term “hydroxy” herein alone or as part of another group refers to —OH. The term “thioalkoxy” herein alone or as part of another group refers to an alkyl group as defined herein attached to the parent molecular group through a sulfur atom. Examples of thioalkoxy include, but are not limited to, thiomethoxy, thioethoxy, and the like.

According to one embodiment of the present invention, methods are provided for treating a tumor that is responsive to IGF1R inhibition comprising administering to a mammal in need of such treatment an effective amount of an IGF1R inhibitor in combination with an effective amount of an insulin-sensitizer. The insulin-sensitizer may be administered to the mammal prior to, subsequent to, or simultaneously with administration of the IGF1R inhibitor.

The insulin-sensitizers of the present invention include, among others, the biguanides, metformin and phenformin, the glitazones (rosiglitazone, piogliatizone), and other PPAR agonists, PPARα, PPARγ, and PPAR dual agonists, such as those described in U.S. Pat. No. 6,414,002 and WO 02/26729, the disclosures of which are herein incorporated by reference.

According to one aspect of the present invention, the insulin-sensitizer is administered to patients undergoing treatment with IGF1R inhibitors in combination with other known anti-cancer treatments. Such treatments include radiation therapy or treatment with cytostatic or cytotoxic agents, such as for example, but not limited to, DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones, either naturally occurring or synthetic; hormonal agents, such as tamoxifen; thymidilate synthase inhibitors, such as 5-fluorouracil and UFT; anti-metabolites, such as methotrexate, tyrosine kinase inhibitors such as Iressa and OSI-774; angiogenesis inhibitors; EGF inhibitors; VEGF inhibitors; CDK inhibitors; Her1/2 inhibitors and monoclonal antibodies directed against growth factor receptors such as erbitux (EGF) and herceptin (Her2), SRC, C-Kit.

IGF1R inhibitors of the present invention include, but are not limited to, those described in U.S. patent application Ser. No. 10/105,599; U.S. patent application Ser. No. 10/263,448; U.S. patent application Ser. No. 10/751,798; U.S. patent application Ser. No. 10/674,098; WO03/048133; WO 01/25220; U.S. Pat. No. 6,337,338 (WO 00/35455); WO 02/102804; WO 02/092599; WO 03/024967; WO 03/035619; WO 03/035616; WO US 2004/63705 and US 2003/125346 the disclosures of which are herein incorporated by reference in their entirety.

In some embodiments of the present invention, IGF1R inhibitors have the following formula I: 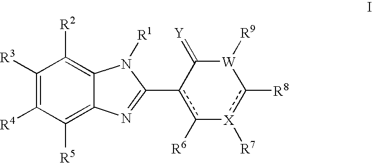
and includes its enantiomers, diastereomers, pharmaceutically acceptable salts, hydrates and solvates thereof;
wherein
X is N, C or a direct bond;
Y is O or S;
W is N, C, O, or S; provided that if W is O or S, R⁹ is absent;
R¹ is H, alkyl, or alkoxy;
R² and R⁹ are independently H or alkyl;
R³ is H, C₁-6 alkyl, alkenyl, alkynyl, cycloalkyl, heterocycloalkyl, halo, amino, OR⁶⁰, —NO₂, —OH, —SR⁶⁰, —NR⁶⁰R⁶⁰, —CN, —C(O)R⁶⁰, —CO₂R⁶⁰, —CONR⁶⁰R⁶¹, OCONR⁶⁰R⁶¹, —NR⁶²CONR⁶⁰R⁶¹, —NR⁶⁰SO₂R⁶¹, —SO₂NR⁶⁰R⁶¹, —SO₂R⁶³, —C(NR⁶²)NR⁶⁰R⁶¹, —C(NH⁶²)-morpholine, aryl, heteroaryl, —(CH₂)ₙC(O)₂—R⁶⁰, NR⁶⁰R⁶¹—(CH₂)ₙOR⁶⁰, —(CH₂)ₙNR⁶⁰ R⁶¹, —(CH₂)ₙSR⁶⁰, —(CH₂)ₙaryl, —(CH₂)ₙ heteroaryl, or —(CH₂)ₙ heterocycloalkyl, wherein n is 1 to 3:
R⁴ is H, halo, alkyl or haloalkyl;
R⁵ is H, alkyl, halo, or aryl;
R⁶, R⁷, and R⁸ are each independently —NH-Z-aryl or —NH-Z-heteroaryl wherein Z is C₁-C₄ alkyl, alkenyl, or alkynyl; Z optionally having one or more hydroxy, thiol, alkoxy, thioalkoxy, amino, halo, NR⁶⁰SO₂R⁶¹ groups; Z optionally incorporating one or more groups selected from the group consisting of CO, CNOH, CNOR⁶⁰, CNNR⁶⁰, CNNCOR⁶⁰ and CNNSO₂R⁶⁰;
R⁶⁰, R⁶¹, R⁶², and R⁶³ are independently selected from the group consisting of H, alkyl, alkenyl, alkynyl, cycloalkyl, cycloalkylalkyl, hydroxy, alkoxy, aryl, heteroaryl, heteroarylalkyl, and alkyl-R¹⁴;
R²⁵ is hydrogen, alkenyl, hydroxy, thiol, alkoxy, thioalkoxy, amino, alkylamino, dialkylamino, aryl, heteroaryl, cyano, halo, sulfoxy, sulfonyl, —NR³⁰COOR³¹, —NR³⁰C(O)R³¹, —NR³⁰SO₂R³¹, —C(O)NR³⁰R³¹R³¹, heteroaryl or heterocycloalkyl; and

R³⁰ and R³¹ are, independently, hydrogen, alkyl, or cycloalkyl.

In some embodiments of the present invention, R¹ is H, alkyl or alkoxy, R² is H; R³ is H, alkyl, —CN, halo, —C(O)R⁶⁰—C(O)NR⁶⁰OR⁶¹, —S(O)₂R⁶³, piperazine, piperidine, morpholine, triazole, imidazole, wherein the piperazine, piperidine, morpholine, triazole, or imidazole is substituted with H, alkyl, —NHC(O)alkyl, —NHC(O)₂alkyl, —NHC(O)alkoxy, —O—(CH₂)ₙR⁶⁴ wherein R⁶⁴ is hydroxy, alkoxy, morpholine, or tetrahydropyrimidine; and R⁶ is —NH-Z-phenyl; —NH-Z-imidazole; or —NH-Z-pyrazole wherein Z is C1 to C2 alkyl.

In a preferred embodiment, R³ is piperidine, morpholine or piperazine.

In some embodiments of the present invention, the IGF1R inhibitor is selected from the group consisting of:
(S)-4-(2-Hydroxy-1-phenyl-ethylamino)-3-(6-imidazol-1-yl-4-methyl-1H-benzimidazol-2-yl)-1H-pyridin-2-one;
(±)-4-[2-Hydroxy-2-(3-iodo-phenyl)-ethylamino]-3-(6-imidazol-1-yl-4-methyl-1H-benzimidazol-2-yl)-1H-pyridin-2-one;
(±)-4-[2-(3-Chloro-phenyl)-2-hydroxy-ethylamino]-3-(6-imidazol-1-yl-4-methyl-1H-benzimidazol-2-yl)-1H-pyridin-2-one;
(±)-4-[2-(3-Bromo-phenyl)-2-hydroxy-ethylamino]-3-(6-imidazol-1-yl-4-methyl-1H-benzimidazol-2-yl)-1H-pyridin-2-one;
(S)-4-[2-(2-Chloro-phenyl)-1-hydroxymethyl-ethylamino]-3-(6-imidazol-1-yl-4-methyl-1H-benzimidazol-2-yl)-1H-pyridin-2-one;
(S)-4-[2-(3-Chloro-phenyl)-1-hydroxymethyl-ethylamino]-3-(6-imidazol-1-yl-4-methyl-1H-benzimidazol-2-yl)-1H-pyridin-2-one;
(S)-4-[2-(4-Chloro-phenyl)-1-hydroxymethyl-ethylamino]-3-(6-imidazol-1-yl-4-methyl-1H-benzimidazol-2-yl)-1H-pyridin-2-one;
(S)-4-[2-(2-Bromo-phenyl)-1-hydroxymethyl-ethylamino]-3-(6-imidazol-1-yl-4-methyl-1H-benzimidazol-2-yl)-1H-pyridin-2-one;
(S)-4-[2-(3-Bromo-phenyl)-1-hydroxymethyl-ethylamino]-3-(6-imidazol-1-yl-4-methyl-1H-benzimidazol-2-yl)-1H-pyridin-2-one;
(±)-4-(1-Hydroxymethyl-2-pentafluorophenyl-ethylamino)-3-(6-imidazol-1-yl-4-methyl-1H-benzimidazol-2-yl)-1H-pyridin-2-one;
(S)-4-(1-Hydroxymethyl-2-pyridin-4-yl-ethylamino)-3-(6-imidazol-1-yl-4-methyl-1H-benzimidazol-2-yl)-1H-pyridin-2-one;
(S)-4-[1-Hydroxymethyl-2-(2-naphthalenyl)-ethylamino]-3-(6-imidazol-1-yl-4-methyl-1H-benzimidazol-2-yl)-1H-pyridin-2-one;
3-(6-Imidazol-1-yl-4-methyl-1H-benzimidazol-2-yl)-4-(pyridin-2-ylmethoxy)-1H-pyridin-2-one;
(±)-4-[2-(3-Bromo-phenyl)-2-fluoro-ethylamino]-3-(6-imidazol-1-yl-4-methyl-1H-benzimidazol-2-yl)-1H-pyridin-2-one;
(S)-2-[4-(1-Hydroxymethyl-2-phenyl-ethylamino)-2-oxo-1,2-dihydro-pyridin-3-yl]-7-methyl-3H-benzimidazole-5-carbonitrile;
(±)-2-{4-[2-(3-Chloro-phenyl)-2-hydroxy-ethylamino]-2-oxo-1,2-dihydro-pyridin-3-yl}-7-methyl-3H-benzimidazole-5-carbonitrile;
(S)-2-{4-[2-(3-Chloro-phenyl)-1-hydroxymethyl-ethylamino]-2-oxo-1,2-dihydro-pyridin-3-yl}-7-methyl-3H-benzimidazole-5-carbonitrile;
(±)-2-{4-[2-(3-Bromo-4-methoxy-phenyl)-2-hydroxy-ethylamino]-2-oxo-1,2-dihydro-pyridin-3-yl}-7-methyl-3H-benzimidazole-5-carbonitrile;
(±)-2-{4-[2-(3-Fluoro-phenyl)-2-hydroxy-ethylamino]-2-oxo-1,2-dihydro-pyridin-3-yl}-7-methyl-3H-benzimidazole-5-carbonitrile;
(±)-2-{4-[2-(3-Bromo-phenyl)-2-hydroxy-ethylamino]-2-oxo-1,2-dihydro-pyridin-3-yl}-7-methyl-3H-benzimidazole-5-carbonitrile;
(S)-2-[4-(2-Hydroxy-2-phenyl-ethylamino)-2-oxo-1,2-dihydro-pyridin-3-yl]-7-methyl-3H-benzimidazole-5-carbonitrile;
(±)-3-(1H-Benzimidazol-2-yl)-4-[2-(3-bromo-phenyl)-2-hydroxy-ethylamino]-1H-pyridin-2-one;
(S)-3-(1H-Benzimidazol-2-yl)-4-(1-hydroxymethyl-2-phenyl-ethylamino)-1H-pyridin-2-one;
(±)-3-(1H-Benzimidazol-2-yl)-4-[2-(3-bromo-4-methoxy-phenyl)-2-hydroxy-ethylamino]-1H-pyridin-2-one;
(S)-4-{2-[4-(1-hydroxymethyl-2-phenyl-ethylamino)-2-oxo-1,2-dihydro-pyridin-3-yl]-7-methyl-3H-benzimidazol-5-yl}-piperazine-1-carboxylic acid isopropylamide;
(S)-4-{2-[4-(1-hydroxymethyl-2-phenyl-ethylamino)-2-oxo-1,2-dihydro-pyridin-3-yl]-7-methyl-3H-benzimidazol-5-yl}-piperazine-1-carboxylic acid ethylamide;
(S)-4-(1-Hydroxymethyl-2-phenyl-ethylamino)-3-{4-methyl-6-[4-(1-phenyl-methanoyl)-piperazin-1-yl]-1H-benzimidazol-2-yl}-1H-pyridin-2-one;
(S)-4-(1-Hydroxymethyl-2-phenyl-ethylamino)-3-[6-(4-isopropyl-piperazin-1-yl)-4-methyl-1H-benzimidazol-2-yl]-1H-pyridin-2-one;
(S)-3-[6-(4-Benzyl-piperazine-1-yl)-4-methyl-1H-benzimidazol-2-yl]-4-(1-hydroxymethyl-2-phenyl-ethylamino)-1H-pyridin-2-one;
(±)-3-[6-(4-Acetyl-piperazine-1-yl)-4-methyl-1H-benzimidazol-2-yl]-4-[2-(3-chloro-phenyl)-2-hydroxy-ethylamino]-1H-pyridin-2-one;
(±)-4-[2-(3-Chloro-phenyl)-2-hydroxy-ethylamino]-3-(4-methyl-6-piperazin-1-yl-1H-benzimidazol-2-yl)-1H-pyridin-2-one;
(±)-4-[2-(3-Chloro-phenyl)-2-hydroxy-ethylamino]-3-[6-(4-isopropyl-piperazine-1-yl)-4-methyl-1H-benzimidazol-2-yl]-1H-pyridin-2-one;
(S)-6-(1-Hydroxymethyl-2-phenyl-ethylamino)-5-(6-imidazol-1-yl-4-methyl-1H-benzimidazol-2-yl)-3H-pyrimidin-4-one;
(S)-2-[6-Chloro-5-(6-imidazol-1-yl-4-methyl-1H-benzimidazol-2-yl)-pyrimidin-4-ylamino]-3-phenyl-propan-1-ol;
(S)-4-(2-Hydroxy-2-phenyl-ethylamino)-3-(6-imidazol-1-yl-4-methyl-1H-benzimidazol-2-yl)-1H-pyridin-2-one;
(R)-4-(2-Hydroxy-2-phenyl-ethylamino)-3-(6-imidazol-1-yl-4-methyl-1H-benzimidazol-2-yl)-1H-pyridin-2-one;
(1S,2R)-4-(1-Hydroxy-indan-2-ylamino)-3-(6-imidazol-1-yl-4-methyl-1H-benzimidazol-2-yl)-1H-pyridin-2-one;
(±)-4-[2-Hydroxy-2-(3-hydroxy-phenyl)-ethylamino]-3-(6-imidazol-1-yl-4-methyl-1H-benzimidazol-2-yl)-1H-pyridin-2-one;
(S)-4-(2-Hydroxy-2-pyridin-2-yl-ethylamino)-3-(6-imidazol-1-yl-4-methyl-1H-benzimidazol-2-yl)-1H-pyridin-2-one;
(±)-N-(3-{1-Hydroxy-2-[3-(6-imidazol-1-yl-4-methyl-1H-benzimidazol-2-yl)-2-oxo-1,2-dihydro-pyridin-4-ylamino]-ethyl}-phenyl)-methanesulfonamide;
(±)-4-[2-(3-Fluoro-phenyl)-2-hydroxy-ethylamino]-3-(6-imidazol-1-yl-4-methyl-1H-benzimidazol-2-yl)-1H-pyridin-2-one;
(±)-4-[2-(3-Chloro-4-fluoro-phenyl)-2-hydroxy-ethylamino]-3-(6-imidazol-1-yl-4-methyl-1H-benzimidazol-2-yl)-1H-pyridin-2-one;
(S)-4-[2-(3-Fluoro-phenyl)-1-hydroxymethyl-ethylamino]-3-(6-imidazol-1-yl-4-methyl-1H-benzimidazol-2-yl)-1H-pyridin-2-one;
(±)-4-[2-(3-Chloro-phenyl)-2-hydroxy-ethylamino]-3-(6-imidazol-1-yl-1H-benzimidazol-2-yl)-1H-pyridin-2-one;
(±)-4-[2-(3-Bromo-4-methoxy-phenyl)-2-hydroxy-ethylamino]-3-(6-imidazol-1-yl-4-methyl-1H-benzimidazol-2-yl)-1H-pyridin-2-one;
(S)-4-[2-(3-Bromo-phenyl)-2-hydroxy-ethylamino]-3-(6-imidazol-1-yl-4-methyl-1H-benzimidazol-2-yl)-1H-pyridin-2-one;
(S)-4-[2-(3-Chloro-phenyl)-2-hydroxy-ethylamino]-3-(6-imidazol-1-yl-4-methyl-1H-benzimidazol-2-yl)-1H-pyridin-2-one;
(R)-4-[2-(3-Chloro-phenyl)-2-hydroxy-ethylamino]-3-(6-imidazol-1-yl-4-methyl-1 H-benzimidazol-2-yl)-1H-pyridin-2-one;
(±)-4-[2-(3-Chloro-4-methoxy-phenyl)-2-hydroxy-ethylamino]-3-(6-imidazol-1-yl-4-methyl-1H-benzimidazol-2-yl)-1H-pyridin-2-one;
(±)-(2-Chloro-4-{1-hydroxy-2-[3-(6-imidazol-1-yl-4-methyl-1H-benzimidazol-2-yl)-2-oxo-1,2-dihydro-pyridin-4-ylamino]-ethyl}-phenyl)-carbamic acid methyl ester;
(S)-4-(1-Hydroxymethyl-2-phenyl-ethylamino)-3-[4-methyl-6-(4-methyl-piperazin-1-yl)-1H-benzimidazol-2-yl]-1H-pyridin-2-one;
(S)-4-(1-Hydroxymethyl-2-phenyl-ethylamino)-3-[4-methyl-6-(4-n-butyl-piperazin-1-yl)-1H-benzimidazol-2-yl]-1H-pyridin-2-one;
(S)-3-{6-[4-(2-Hydroxy-ethyl)-piperazin-1-yl]-4-methyl-1H-benzimidazol-2-yl}-4-(1-hydroxymethyl-2-phenyl-ethylamino)-1H-pyridin-2-one;
(S)-4-{2-[4-(1-Hydroxymethyl-2-phenyl-ethylamino)-2-oxo-1,2-dihydro-pyridin-3-yl]-7-methyl-3H-benzimidazol-5-yl}-piperazine-1-carboxylic acid amide;
(±)-4-[2-(3-Chloro-phenyl)-2-hydroxy-ethylamino]-3-(4-methyl-6-piperazin-1′-yl-1H-benzimidazol-2-yl)-1H-pyridin-2-one;
(±)-4-[2-(3-Chloro-phenyl)-2-hydroxy-ethylamino]-3-[6-(4-ethyl-piperazin-1-yl)-4-methyl-1H-benzimidazol-2-yl]-1H-pyridin-2-one;
(±)-4-[2-(3-Chloro-phenyl)-2-hydroxy-ethylamino]-3-{6-[4-(2-hydroxy-ethyl)piperazin-1-yl]-4-methyl-1H-benzimidazol-2-yl}-1H-pyridin-2-one;
(±)-4-[2-(3-Chloro-phenyl)-2-hydroxy-ethylamino]-3-(4-methyl-6-morpholin-4-yl-1H-benzimidazol-2-yl)-1H-pyridin-2-one;
(±)-4-[2-(3-Bromo-phenyl)-2-hydroxy-ethylamino]-3-(4-methyl-6-morpholin-4-yl-1H-benzimidazol-2-yl)-1H—;
(±)-4-[2-(3-Bromo-4-methoxy-phenyl)-2-hydroxy-ethylamino]-3-(4-methyl-6-morpholin-4-yl-1H-benzimidazol-2-yl)-1H-pyridin-2-one;
(±)-4-[2-(3-Bromo-phenyl)-2-hydroxy-ethylamino]-3-{6-[4-(2-hydroxy-ethyl)-piperazin-1-yl]-4-methyl-1H-benzimidazol-2-yl})-1H-pyridin-2-one;
(±)-4-[2-(3-Bromo-phenyl)-2-hydroxy-ethylamino]-3-(4-methyl-6-piperazin-1-yl-1H-benzimidazol-2-yl}-1H-pyridin-2-one;
(±)-4-[2-(3-Bromo-phenyl)-2-hydroxy-ethylamino]-3-(4-methyl-6-piperazin-1-yl-1H-benzimidazol-2-yl}-1H-pyridin-2-one;
(±)-3-[6-(4-Acetyl-piperazin-1-yl)-4-methyl-1H-benzimidazol-2-yl]-4-[2-(3-bromo-phenyl)-2-hydroxy-ethylamino]-1H-pyridin-2-one;
(S)-4-(1-hydroxymethyl-2-phenyl-ethylamino)-3-[4-methyl-6-(2-morpholin-4-yl-ethylamino)-1H-benzimidazol-2-yl]-1H-pyridin-2-one;
(±)-6-[2-(3-Chloro-phenyl)-2-hydroxy-ethylamino]-5-(6-imidazol-1-yl-4-methyl-1H-benzimidazol-2-yl)-3H-pyrimidin-4-one;
(±)-4-[2-(3-Chloro-phenyl)-2-hydroxy-ethylamino]-3-[6-(1-hydroxy-1-methyl-ethyl)-4-methyl-1H-benzimidazol-2-yl]-1H-pyridin-2-one;
(±)-3-(6-Aminomethyl-4-methyl-1H-benzimidazol-2-yl)-4-[2-(3-chloro-phenyl)-2-hydroxy-ethylamino]-1H-pyridin-2-one;
(±)-4-[2-(3-Chloro-phenyl)-2-hydroxy-ethylamino]-3-(6-hydroxymethyl-4-methyl-1H-benzimidazol-2-yl)-1H-pyridin-2-one;
(S)-4-(1-Benzyl-2-hydroxy-ethylamino)-3-(4-methyl-6-morpholin-4-yl-1H-benzimidazol-2-yl)-1H-pyridin-2-one; and
(S)-4-(1-Benzyl-2-hydroxy-ethylamino)-3-(4-methyl-6-piperidin-1-yl-1H-benzimidazol-2-yl)-1H-pyridin-2-one;
(S)-4-(1-Benzyl-2-hydroxy-ethylamino)-3-(4-methyl-6-piperidin-1-yl-1H-benzimidazol-2-yl)-1H-pyridin-2-one;
4-[2-(3-Chloro-4-methylsulfanyl-phenyl)-2-hydroxy-ethylamino]-3-(4-methyl-6-piperazin-1-yl-1H-benzoimidazol-2-yl)-1H-pyridin-2-one;
4-[2-(3-Chloro-4-fluoro-phenyl)-2-hydroxy-ethylamino]-3-(4-methyl-6-piperazin-1-yl-1H-benzoimidazol-2-yl)-1H-pyridin-2-one;
3-[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-propionitrile;
4-[2-(3-Chloro-phenyl)-2-hydroxy-ethylamino]-3-{6-[4-(2-methanesulfonyl-ethyl)-piperazin-1-yl]-4-methyl-1H-benzoimidazol-2-yl}-1H-pyridin-2-one;
3-[4-(2-{4-[2-(3-Bromo-4-methoxy-phenyl)-2-hydroxy-ethylamino]-2-oxo-1,2-dihydro-pyridin-3-yl}-3H-benzoimidazol-5-yl)-7-methyl-piperazin-1-yl]-propionitrile;
4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid 2-fluoro-ethyl ester;
4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid 2-methoxy-ethyl ester;
4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid tert-butyl ester;
4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid prop-2-ynyl ester;
4-(2-{4-[2-(3-Bromo-4-methoxy-phenyl)-2-hydroxy-ethylamino]-2-oxo-1,2-dihydro-pyridin-3-yl}-7-methyl-3H-benzoimidazol-5-yl)-piperazine-1-carboxylic acid tert-butyl ester;
(S)-4-(2-{4-[2-(3-Bromo-4-methoxy-phenyl)-2-hydroxy-ethylamino]-2-oxo-1,2-dihydro-pyridin-3-yl}-7-methyl-3H-benzimidazol-5-yl)-piperazine-1-carboxylic acid ethyl ester;
4-[2-(3-Chloro-4-methoxy-phenyl)-2-hydroxy-ethylamino]-3-{6-[4-(3-fluoro-propyl)-piperazin-1-yl]-4-methyl-1H-benzoimidazol-2-yl}-1H-pyridin-2-one;
4-[2-(3-Chloro-phenyl)-2-hydroxy-ethylamino]-3-{6-[4-(2-fluoro-ethyl)-piperazin-1-yl]-4-methyl-1H-benzoimidazol-2-yl}-1H-pyridin-2-one;
4-[2-(3-Chloro-4-fluoro-phenyl)-2-hydroxy-ethylamino]-3-{6-[4-(3-fluoro-propyl)-piperazin-1-yl]-4-methyl-1H-benzoimidazol-2-yl}-1H-pyridin-2-one;
4-[2-(3-Bromo-4-methoxy-phenyl)-2-hydroxy-ethylamino]-3-{6-[4-(3-fluoro-propyl)-piperazin-1-yl]-4-methyl-1H-benzoimidazol-2-yl}-1H-pyridin-2-one;
4-[2-(3-Chloro-phenyl)-2-hydroxy-ethylamino]-3-{4-methyl-6-[4-(3,3,3-trifluoro-propyl)-piperazin-1-yl]-1H-benzoimidazol-2-yl}-1H-pyridin-2-one;
4-[2-(3-Chloro-phenyl)-2-hydroxy-ethylamino]-3-{6-[4-(3-fluoro-propyl)-piperazin-1-yl]-4-methyl-1H-benzoimidazol-2-yl}-1H-pyridin-2-one;
4-[2-(3-Chloro-phenyl)-2-hydroxy-ethylamino]-3-{4-methyl-6-[4-(3,4,4-trifluoro-but-3-enyl)-piperazin-1-yl]-1H-benzoimidazol-2-yl}-1H-pyridin-2-one;
4-[2-(3-Chloro-phenyl)-2-hydroxy-ethylamino]-3-{6-[4-(3-fluoro-2-hydroxy-propyl)-piperazin-1-yl]-4-methyl-1H-benzoimidazol-2-yl}-1H-pyridin-2-one;
4-[2-(3-Chloro-phenyl)-2-hydroxy-ethylamino]-3-{6-[4-(2-hydroxy-2-methyl-propyl)-piperazin-1-yl]-4-methyl-1H-benzoimidazol-2-yl}-1H-pyridin-2-one;
(S)-4-[2-(3-Chloro-phenyl)-2-hydroxy-ethylamino]-3-{6-[4-(2-hydroxy-ethyl)-piperazin-1-yl]-4-methyl-1H-benzimidazol-2-yl}-1H-pyridin-2-one;
(S)-4-[2-(3-Bromo-4-methoxy-phenyl)-2-hydroxy-ethylamino]-3-{6-[4-(2-hydroxy-ethyl)-piperazin-1-yl]-4-methyl-1H-benzimidazol-2-yl}-1H-pyridin-2-one;
[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-acetonitrile;
4-[2-(3-Chloro-phenyl)-2-hydroxy-ethylamino]-3-{6-[4-(4-fluoro-butyryl)-piperazin-1-yl]-4-methyl-1H-benzoimidazol-2-yl}-1H-pyridin-2-one;
4-[2-(3-Chloro-phenyl)-2-hydroxy-ethylamino]-3-{6-[4-(2,2-difluoro-acetyl)-piperazin-1-yl]-4-methyl-1H-benzoimidazol-2-yl}-1H-pyridin-2-one;
4-[2-(3-Chloro-phenyl)-2-hydroxy-ethylamino]-3-{6-[4-(2-methanesulfonyl-acetyl)-piperazin-1-yl]-4-methyl-1H-benzoimidazol-2-yl}-1H-pyridin-2-one;
3-[6-(4-Acetyl-piperazin-1-yl)-4-methyl-1H-benzoimidazol-2-yl]-4-[2-(3-chloro-phenyl)-2-hydroxy-ethylamino]-1H-pyridin-2-one;
4-[2-(3-Chloro-phenyl)-2-hydroxy-ethylamino]-3-(4-methyl-6-{4-[2-(1-oxo-114-thiomorpholin-4-yl)-acetyl]-piperazin-1-yl}-1H-benzoimidazol-2-yl)-1H-pyridin-2-one;
4-[2-(3-Chloro-phenyl)-2-hydroxy-ethylamino]-3-(6-{4-[2-(1, 1-dioxo-116-thiomorpholin-4-yl)-acetyl]-piperazin-1-yl}-4-methyl-1H-benzoimidazol-2-yl)-1H-pyridin-2-one;
4-[2-(3-Chloro-phenyl)-2-hydroxy-ethylamino]-3-{4-methyl-6-[4-(2-thiomorpholin-4-yl-acetyl)-piperazin-1-yl]-1H-benzoimidazol-2-yl}-1H-pyridin-2-one;
4-[2-(3-Chloro-phenyl)-2-hydroxy-ethylamino]-3-{6-[4-(2-methanesulfinyl-acetyl)-piperazin-1-yl]-4-methyl-1H-benzoimidazol-2-yl}-1H-pyridin-2-one;
4-[2-(3-Chloro-phenyl)-2-hydroxy-ethylamino]-3-{6-[4-(2-methoxy-acetyl)-piperazin-1-yl]-4-methyl-1H-benzoimidazol-2-yl}-1H-pyridin-2-one;
4-[2-(3-Chloro-phenyl)-2-hydroxy-ethylamino]-3-{4-methyl-6-[4-(2-methylsulfanyl-acetyl)-piperazin-1-yl]-1H-benzoimidazol-2-yl}-1H-pyridin-2-one;
3-{6-[4-(2-Chloro-acetyl)-piperazin-1-yl]-4-methyl-1H-benzoimidazol-2-yl}-4-[2-(3-chloro-phenyl)-2-hydroxy-ethylamino]-1H-pyridin-2-one;
(S)-4-(2-{4-[2-(3-Bromo-4-methoxy-phenyl)-2-hydroxy-ethylamino]-2-oxo-1,2-dihydro-pyridin-3-yl}-7-methyl-3H-benzimidazol-5-yl)-piperazine-1-carbaldehyde;
(S)-4-(2-{4-[2-(3-Chloro-phenyl)-2-hydroxy-ethylamino]-2-oxo-1,2-dihydro-pyridin-3-yl}-7-methyl-3H-benzimidazol-5-yl)-piperazine-1-carbaldehyde;
(S)-4-[2-(3-Chloro-phenyl)-2-hydroxy-ethylamino]-3-(4-methyl-6-morpholin-4-yl-1H-benzoimidazol-2-yl)-1H-pyridin-2-one;
4-[2-(3-Bromo-4-methoxy-phenyl)-2-hydroxy-ethylamino]-3-(4-methyl-6-morpholin-4-yl-1H-benzoimidazol-2-yl)-1H-pyridin-2-one;
4-[2-(3-Chloro-4-fluoro-phenyl)-2-hydroxy-ethylamino]-3-(4-methyl-6-morpholin-4-yl-1H-benzoimidazol-2-yl)-1H-pyridin-2-one;
4-[2-(3-Chloro-4-methoxy-phenyl)-2-hydroxy-ethylamino]-3-(4-methyl-6-morpholin-4-yl-1H-benzoimidazol-2-yl)-1H-pyridin-2-one;
4-[2-(7-Bromo-2,3-dihydro-benzofuran-5-yl)-2-hydroxy-ethylamino]-3-(4-methyl-6-morpholin-4-yl-1H-benzoimidazol-2-yl)-1H-pyridin-2-one;
4-[2-(3-Chloro-phenyl)-2(S)-hydroxy-ethylamino]-3-[4-methyl-6-[2(S),6(R)-dimethyl-morpholine-4-yl]-1H-benzoimidazol-2-yl]-1H-pyridine-2-one;
4-[2-(3-Bromo-4-methoxy-phenyl)-2(S)-hydroxy-ethylamino]-3-[4-methyl-6-[2(S),6(R)-dimethyl-morpholine-4-yl]-1H-benzoimidazol-2-yl]-1H-pyridine-2-one;
4-[2-(3-Chloro-phenyl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-fluoromethyl-morpholin-4-yl]-4-methyl-1H-benzimidazol-2-yl}-1H-pyridin-2-one;
4-[2-(3-Bromo-4-methoxy-phenyl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-fluoromethyl-morpholin-4-yl]-4-methyl-1H-benzimidazol-2-yl}-1H-pyridin-2-one;
4-[2-(3-Chloro-4-methoxy-phenyl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-fluoromethyl-morpholin-4-yl]-4-methyl-1H-benzimidazol-2-yl}-1H-pyridin-2-one;
4-[2-(7-Bromo-2,3-dihydro-benzofuran-4-yl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(7-bromo-2,3-dihydro-benzofuran-4-yl)-(S)-2-hydroxy-ethylamino]-3-{6-[(S)-2-fluoromethyl-morpholin-4-yl]-4-methyl-1H-benzimidazol-2-yl}-1H-pyridin-2-one;
4-[2-(3-Chloro-phenyl)-(S)-2-hydroxy-ethylamino]-3-{6-[(R)-2-hydroxymethyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-hydroxy-methyl-morpholin-4-yl]-4-methyl-1H-benzimidazol-2-yl}-1H-pyridin-2-one;
4-[2-(3-Bromo-4-methoxy-phenyl)-(S)-2-hydroxy-ethylamino]-3-{6-[(R)-2-hydroxymethyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-hydroxy-methyl-morpholin-4-yl]-4-methyl-1H-benzimidazol-2-yl}-1H-pyridin-2-one;
4-[2-(3-Chloro-4-methoxy-phenyl)-(S)-2-hydroxy-ethylamino]-3-{6-[(R)-2-hydroxymethyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-hydroxy-methyl-morpholin-4-yl]-4-methyl-1H-benzimidazol-2-yl}-1H-pyridin-2-one;
4-[2-(3-Chloro-phenyl)-(S)-2-hydroxy-ethylamino]-3-{6-[(R)-2-methyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-methyl-morpholin-4-yl]-4-methyl-1H-benzimidazol-2-yl}-1H-pyridin-2-one;
4-[2-(3-Bromo-4-methoxy-phenyl)-(S)-2-hydroxy-ethylamino]-3-{6-[(R)-2-methyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-methyl-morpholin-4-yl]-4-methyl-1H-benzimidazol-2-yl}-1H-pyridin-2-one;
4-[2-(3-Chloro-4-methoxy-phenyl)-(S)-2-hydroxy-ethylamino]-3-{6-[(R)-2-methyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-methyl-morpholin-4-yl]-4-methyl-1H-benzimidazol-2-yl}-1H-pyridin-2-one;
4-[2-(3-Chloro-phenyl)-(S)-2-hydroxy-ethylamino]-3-{6-[(R)-2-methoxymethyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-methoxy-methyl-morpholin-4-yl]-4-methyl-1H-benzimidazol-2-yl}-1H-pyridin-2-one;
4-[2-(3-Bromo-4-methoxy-phenyl)-(S)-2-hydroxy-ethylamino]-3-{6-[(R)-2-methoxymethyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-methoxymethyl-morpholin-4-yl]-4-methyl-1H-benzimidazol-2-yl}-1H-pyridin-2-one;
4-[2-(3-Chloro-4-methoxy-phenyl)-(S)-2-hydroxy-ethylamino]-3-{6-[(R)-2-methoxymethyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-methoxymethyl-morpholin-4-yl]-4-methyl-1H-benzimidazol-2-yl}-1H-pyridin-2-one;
4-[2-(3-Chloro-phenyl)-2(S)-hydroxy-ethylamino]-3-[4-methyl-6-(4-methyl-piperazin-1-yl)-1H-benzoimidazol-2-yl]-1H-pyridine-2-one;
4-[2-(3-Bromo-4-methoxy-phenyl)-2(S)-hydroxy-ethylamino]-3-[4-methyl-6-(4-methyl-piperazin-1-yl)-1H-benzoimidazol-2-yl]-1H-pyridine-2-one;
4-[2-(3-Chloro-phenyl)-2-hydroxy-ethylamino]-3-{6-[4-(acetamido)-piperidin-1-yl]-4-methyl-1H-benzoimidazol-2-yl}-1H-pyridin-2-one;
4-[2-(3-Chloro-phenyl)-2-hydroxy-ethylamino]-3-{6-[4-(2-hydroxyacetamido)-piperidin-1-yl]-4-methyl-1H-benzoimidazol-2-yl}-1H-pyridin-2-one;
4-[2-(3-Chloro-phenyl)-2-hydroxy-ethylamino]-3-{6-[4-(2-fluoroacetamido)-piperidin-1-yl]-4-methyl-1H-benzoimidazol-2-yl}-1H-pyridin-2-one;
4-[2-(3-Bromo-4-methoxy-phenyl)-2-hydroxy-ethylamino]-3-{6-[4-(acetamido)-piperidin-1-yl]-4-methyl-1H-benzoimidazol-2-yl}-1H-pyridin-2-one;
4-[2-(3-Bromo-phenyl)-2-hydroxy-ethylamino]-3-{6-[4-(2-hydroxyacetamido)-piperidin-1-yl]-4-methyl-1H-benzoimidazol-2-yl}-1H-pyridin-2-one;
4-[2-(3-Chloro-phenyl)-2-hydroxy-ethylamino]-3-{6-[4-(2-fluoroacetamido)-piperidin-1-yl]-4-methyl-1H-benzoimidazol-2-yl}-1H-pyridin-2-one;
4-[2-(3-Chloro-phenyl)-2-hydroxy-ethylamino]-3-{6-[4-(2-methoxyethoxycarbamoyl)-piperidin-1-yl]-4-methyl-1H-benzoimidazol-2-yl}-1H-pyridin-2-one;
4-[2-(3-Chloro-phenyl)-2-hydroxy-ethylamino]-3-{6-[4-(methoxycarbamoyl)-piperidin-1-yl]-4-methyl-1H-benzoimidazol-2-yl}-1H-pyridin-2-one;
4-[2-(3-Chloro-phenyl)-2-hydroxy-ethylamino]-3-{6-[4-(2-fluoroethoxy carbamoyl)-piperidin-1-yl]-4-methyl-1H-benzoimidazol-2-yl}-1H-pyridin-2-one;
(S)-4-[2-(3-Chloro-phenyl)-2-hydroxy-ethylamino]-3-[4-methyl-6-(2-morpholin-4-yl-ethoxy)-1H-benzimidazol-2-yl]-1H-pyridin-2-one;
(S)-4-[2-(3-Bromo-4-methoxy-phenyl)-2-hydroxy-ethylamino]-3-[4-methyl-6-(2-morpholin-4-yl-ethoxy)-1H-benzimidazol-2-yl]-1H-pyridin-2-one;
(S)-4-[2-(3-Chloro-phenyl)-2-hydroxy-ethylamino]-3-[4-methyl-6-(2-methoxy-ethoxy)-1H-benzimidazol-2-yl]-1H-pyridin-2-one;
(S)-4-[2-(3-Chloro-phenyl)-2-hydroxy-ethylamino]-3-[4-methyl-6-(2-hydroxy-ethoxy)-1H-benzimidazol-2-yl]-1H-pyridin-2-one;
(S)-4-[2-(3-Bromo-4-methoxy-phenyl)-2-hydroxy-ethylamino]-3-[4-methyl-6-(2-morpholin-4-yl-propoxy)-1H-benzimidazol-2-yl]-1H-pyridin-2-one;
(S)-4-[2-(3-Chloro-phenyl)-2-hydroxy-ethylamino]-3-[4-methyl-6-(2-morpholin-4-yl-propoxy)-1H-benzimidazol-2-yl]-1H-pyridin-2-one;
(S)-3-(4-Bromo-6-morpholin-4-ylmethyl-1H-benzimidazol-2-yl)-4-[2-(3-chloro-phenyl)-2-hydroxy-ethylamino]-1H-pyridin-2-one;
(S)-3-[4-Bromo-6-(4-methyl-piperazin-1-ylmethyl-1H-benzimidazol-2-yl)-4-[2-(3-chloro-phenyl)-2-hydroxy-ethylamino]-1H-pyridin-2-one;
(S)-4-[2-(3-Chloro-phenyl)-2-hydroxy-ethylamino]-3-[4-methyl-6-(4-methyl-piperazin-1-ylmethyl)-1H-benzimidazol-2-yl]-1H-pyridin-2-one;
4-[2-(3-Chloro-phenyl)-2(S)-hydroxy-ethylamino]-3-[4-methyl-6-(1,4,5,6-tetrahydropyrimidine-1-yl)-1H-benzoimidazol-2-yl]-1H-pyridine-2-one; and
4-[2-(4-Methoxy-3-Chloro-phenyl)-2(S)-hydroxy-ethylamino]-3-[4-methyl-6-(1,4,5,6-tetrahydropyrimidine-1-yl)-1H-benzoimidazol-2-yl]-1H-pyridine-2-one;
4-[2-(3-Chloro-4-methoxy-phenyl)-2-hydroxy-ethylamino]-3-(4-methyl-6-morpholin-4-yl-1H-benzoimidazol-2-yl)-1,5-dihydro-pyrrol-2-one;
4-[2-(3-Bromo-4-methoxy-phenyl)-2-hydroxy-ethylamino]-3-(4-methyl-6-morpholin-4-yl-1H-benzoimidazol-2-yl)-1,5-dihydro-pyrrol-2-one;
(S)-4-[2-(3-Chloro-phenyl)-2-hydroxy-ethylamino]-3-(4-methyl-6-morpholin-4-yl-1H-benzoimidazol-2-yl)-1,5-dihydro-pyrrol-2-one;
(S, S and S,R)-4-[2-(3-Chloro-phenyl)-2-hydroxy-ethylamino]-5-methyl-3-(4-methyl-6-morpholin-4-yl-1H-benzoimidazol-2-yl)-1,5-dihydro-pyrrol-2-one;
[1-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperidin-4-yl]-carbamic acid tetrahydro-furan-3-ylmethyl ester;
[1-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperidin-4-yl]-carbamic acid 2-methoxy-propyl ester;
(S)-2-[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-acetamide Bis hydrochloride;
(S)-4-[2-(3-Chloro-phenyl)-2-hydroxy-ethylamino]-3-{6[4-(2-methyoxy-ethyl)-piperazin-1-yl]-4-methyl-1H-benzoimidazol-2-yl 1H-pyridin-2-one bis-hydrochloride;
(S)-4-[2-(3-Bromo-phenyl)-2-hydroxy-ethylamino]-3-{6[4-(2-methyoxy-ethyl)-piperazin-1-yl]-4-methyl-1H-benzoimidazol-2-yl-1H-pyridin-2-one bis hydrochloride;
(S)-4-[2-(3-Cynao-phenyl)-2-hydroxy-ethylamino]-3-{6 [4-(2-methyoxy-ethyl)-piperazin-1-yl]-4-methyl-1H-benzoimidazol-2-yl 1H-pyridin-2-one bis hydrochloride;
(S)-4-[2-(3-Chloro-phenyl)-2-hydroxy-ethylamino]-3-{6-[4-(2-hydroxy-ethyl)-piperadin-1-yl]-4-methyl-1H-benzimidazol-2-yl}-1H-pyridin-2-one bis hydrochloride;
(S)-4-[2-(3-Chloro-phenyl)-2-hydroxy-ethylamino]-3-{4-methyl-6-[4-(2-methylsulfanyl-ethyl)-piperazin-1-yl]-1H-benzoimidazol-2-yl}-1H-pyridin-2-one bis hydrochloride;
(S)-4-[2-(3-Chloro-phenyl)-2-hydroxy-ethylamino]-3-[4-methyl-6-(3R-methyl-piperazin-1-yl)-1H-benzoimidazol-2-yl]-1H-pyridin-2-one bis hydrochloride; and
(S)4-[2-(3-Chloro-phenyl)-2-hydroxy-ethylamino]-3-{6-[4-(2-methoxy-ethyl)-3(R)-methyl-piperazin-1-yl]-4-methyl-1H-benzoimidazol-2-yl}-1H-pyridin-2-one bis hydrochloride.

In some embodiments of the present invention, the IGF1R inhibitor has the following formula: 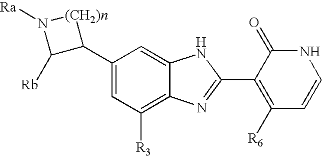
wherein
n is 0 to 4;
Rₐ is alkyl, substituted alkyl, —C(O)pR⁷⁰, aminolalkylamino, —SO₂R⁷¹, cycloalkyl, heterocycloalkyl, heteroaryl, or alkoxyalkoxyalkyl; wherein p is 1 or 2; R⁷⁰ and R⁷¹ are alkyl or substituted alkyl; R_{b} is alkyl or substituted alkyl; and R³ and R⁶ are as defined above. In a preferred embodiment, R⁶ is —NH-Z-heteroaryl or —NH-Z-aryl.

IGF1R inhibitors of the present invention also include the following compounds:

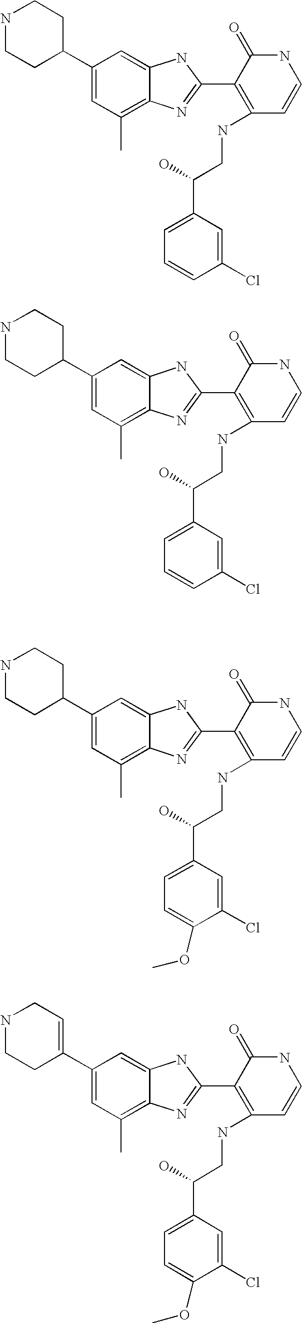

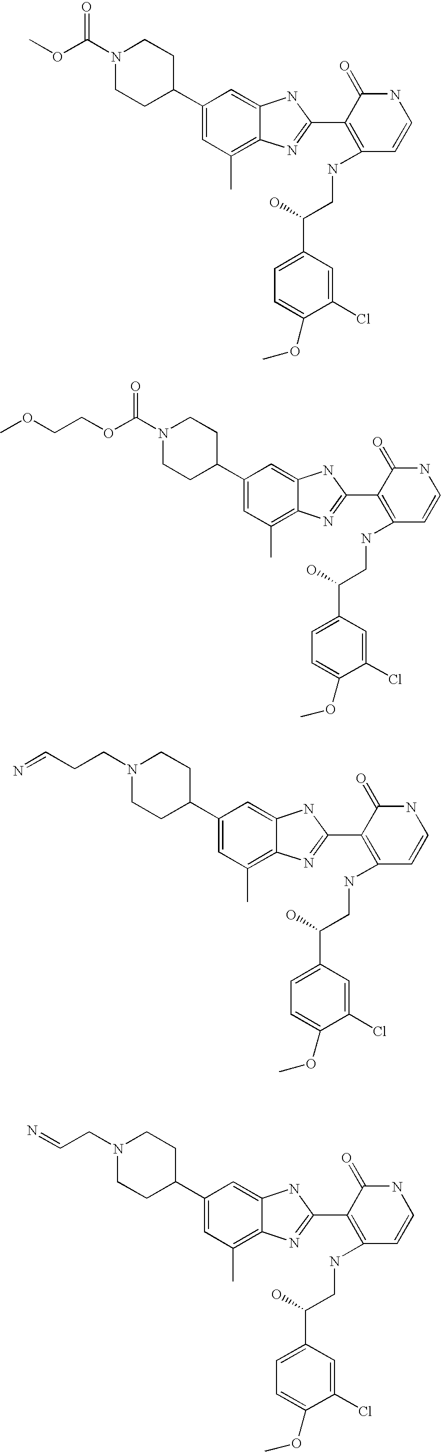

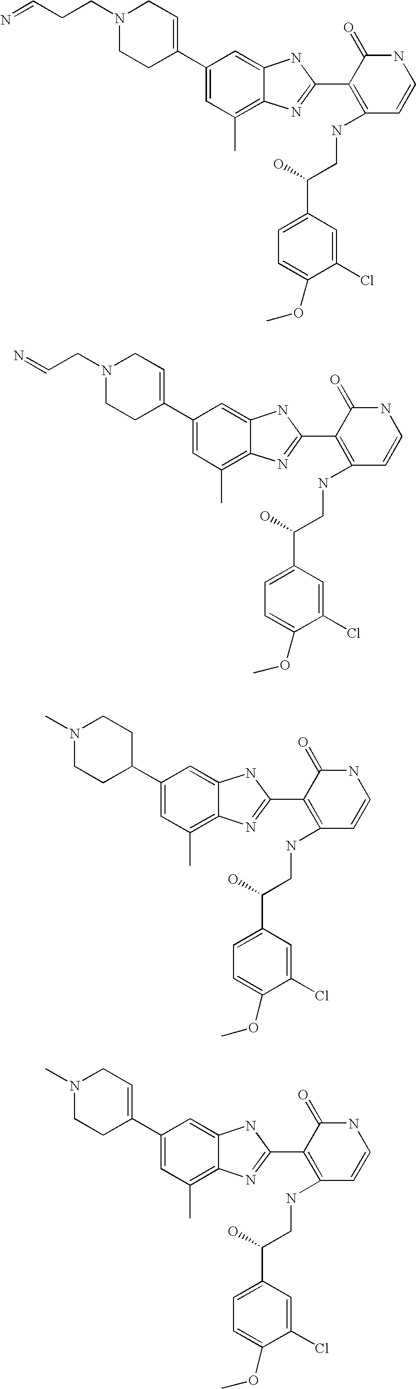

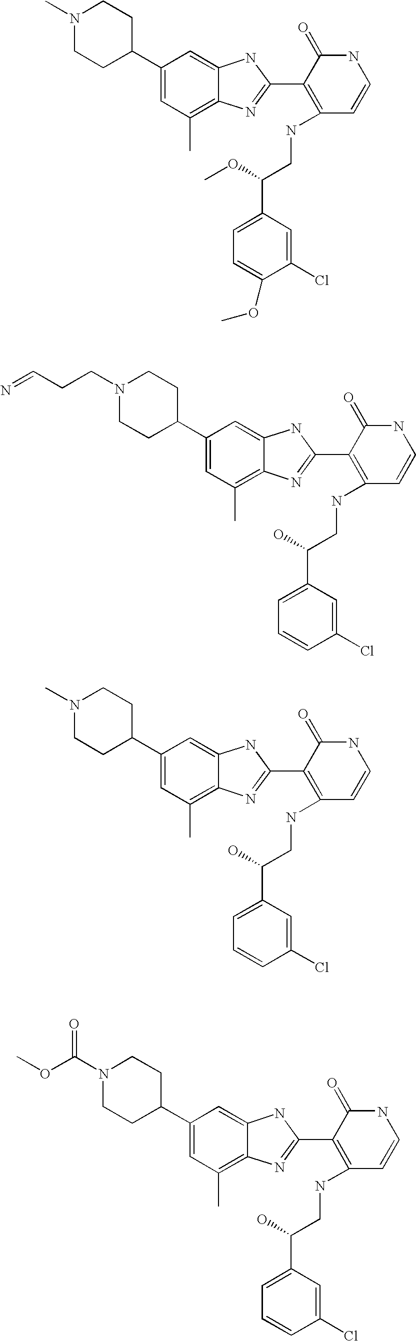

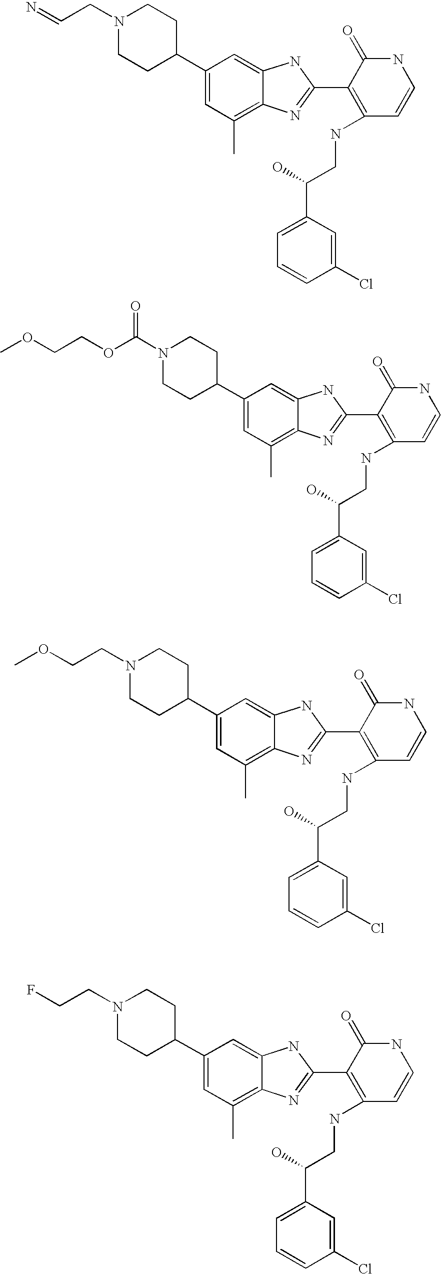

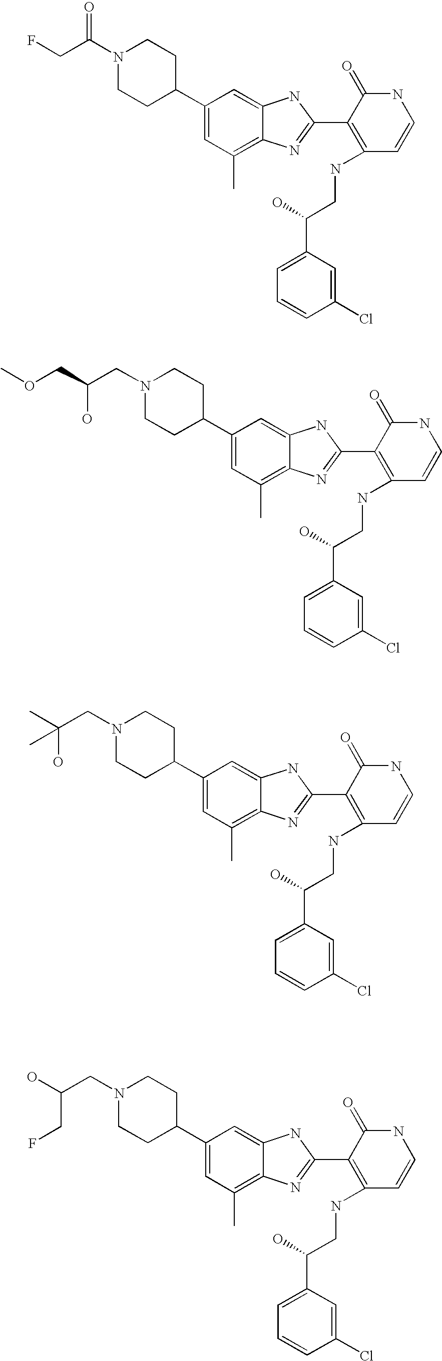

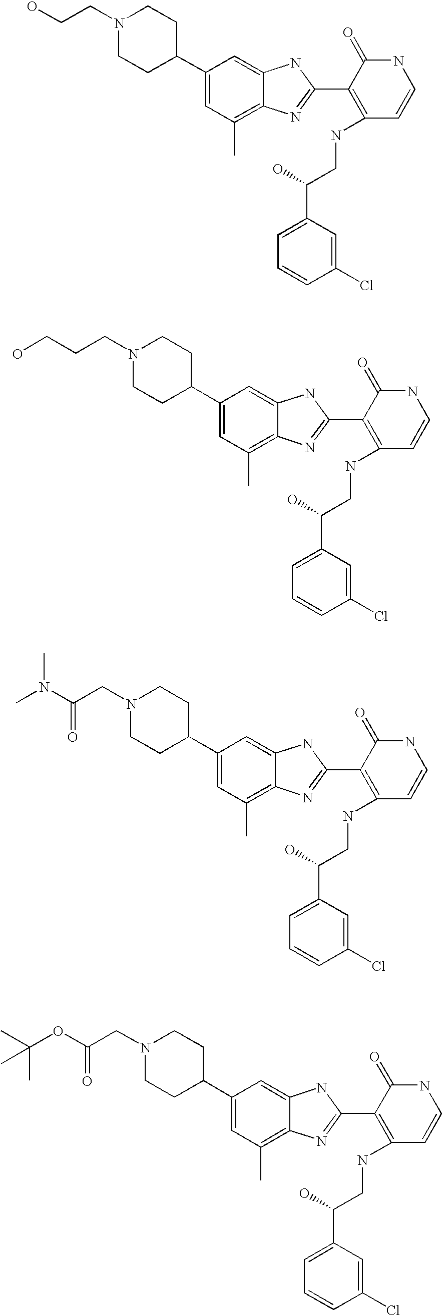

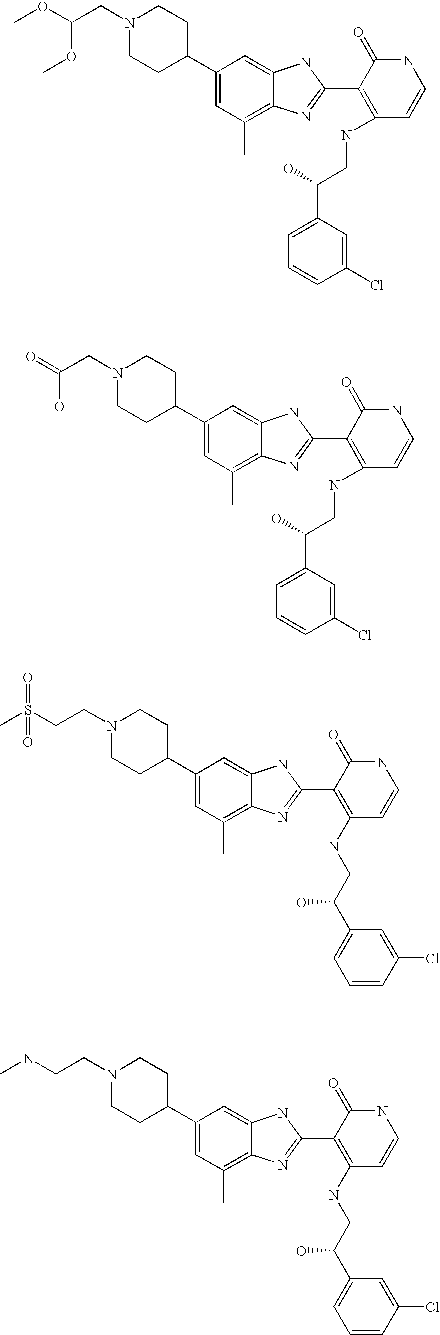

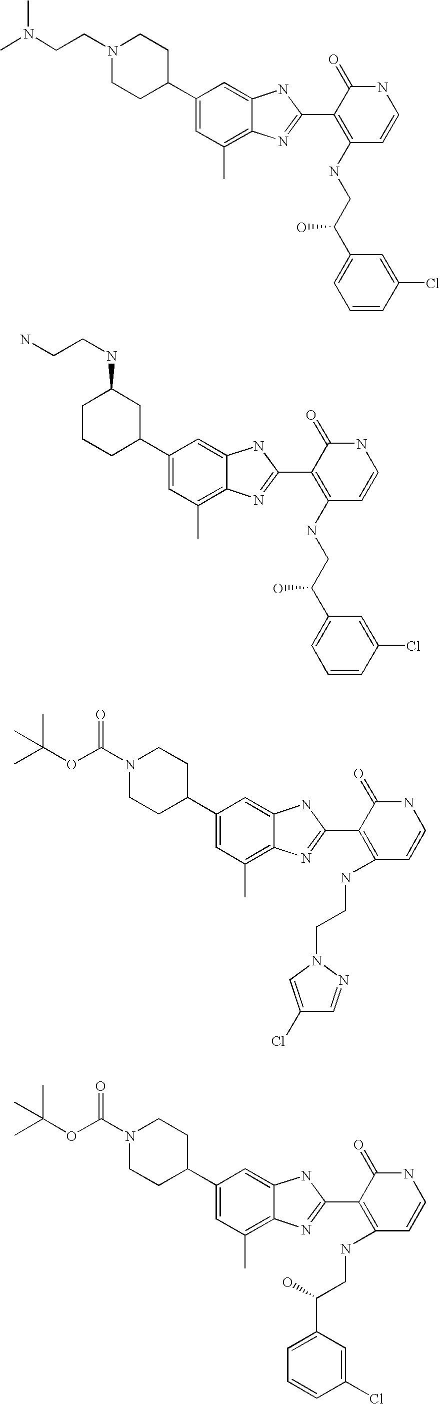

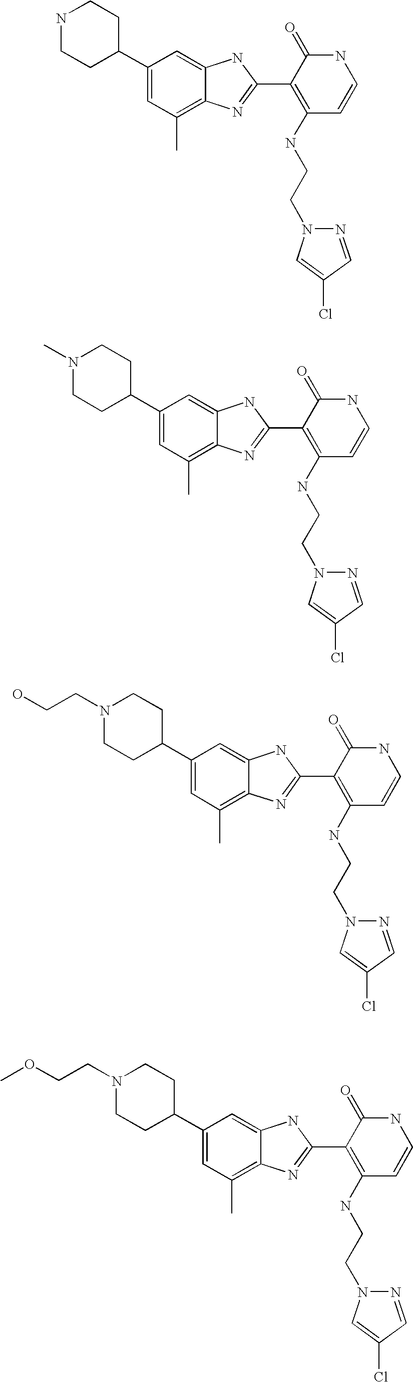

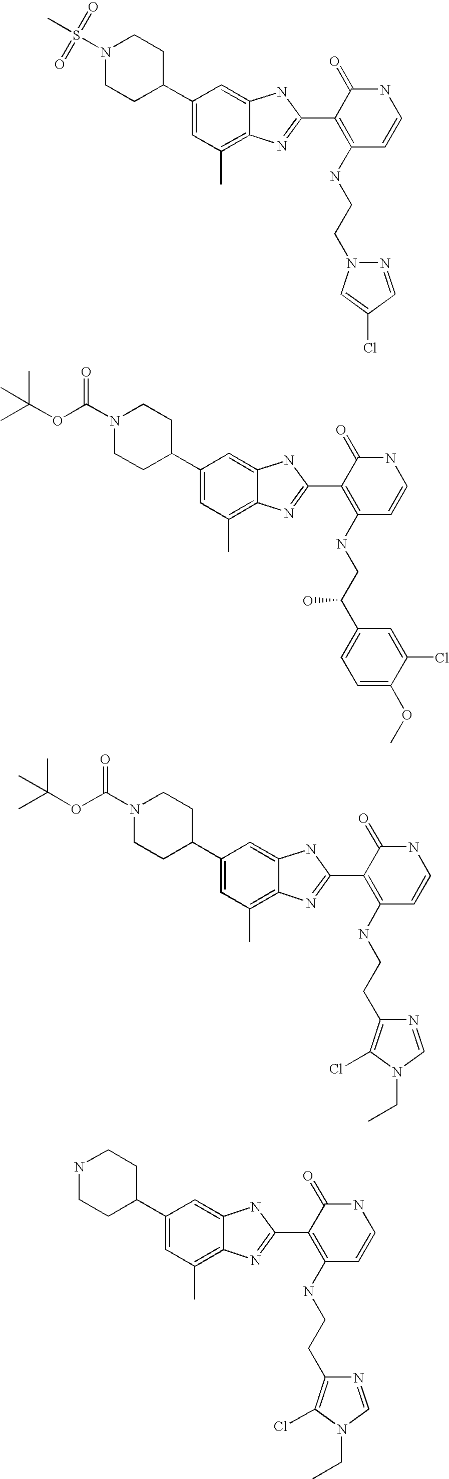

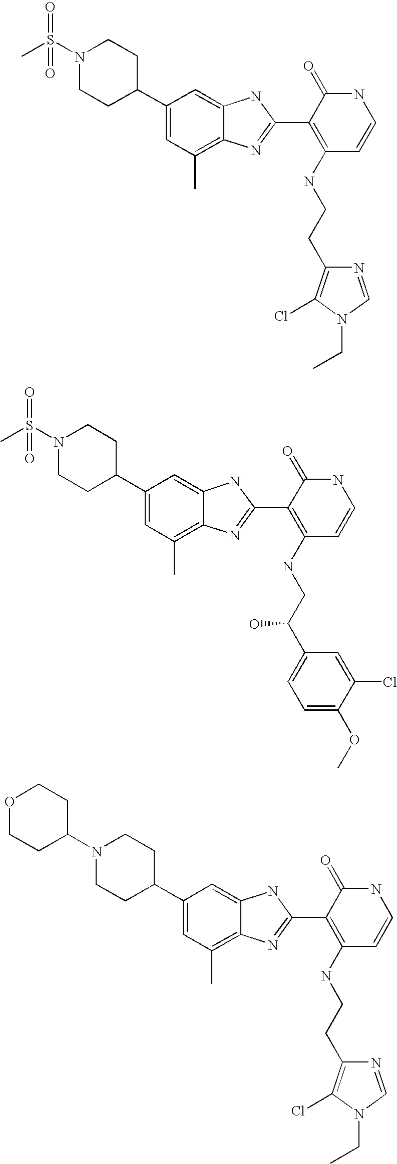

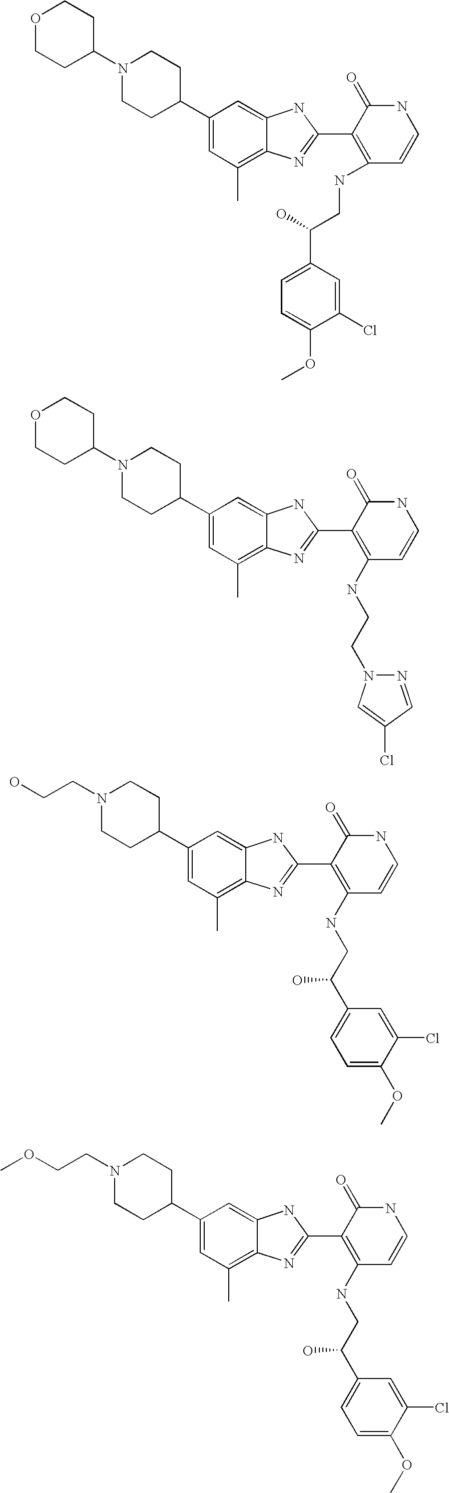

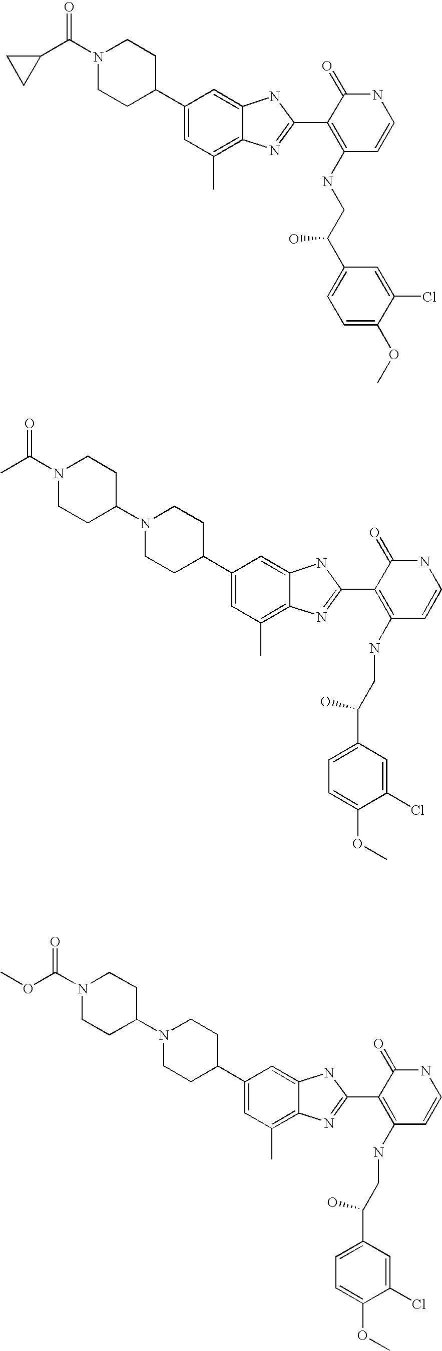

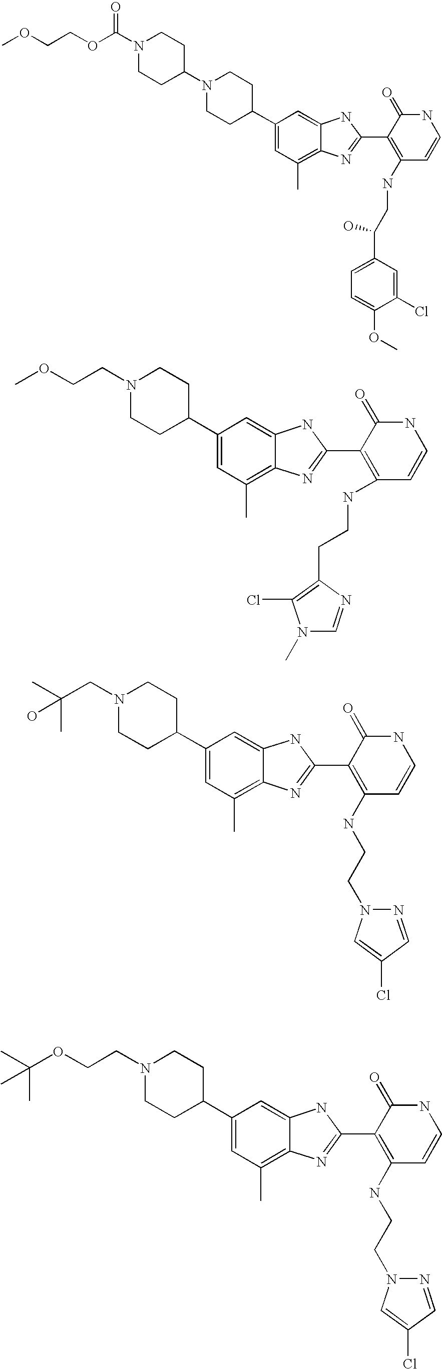

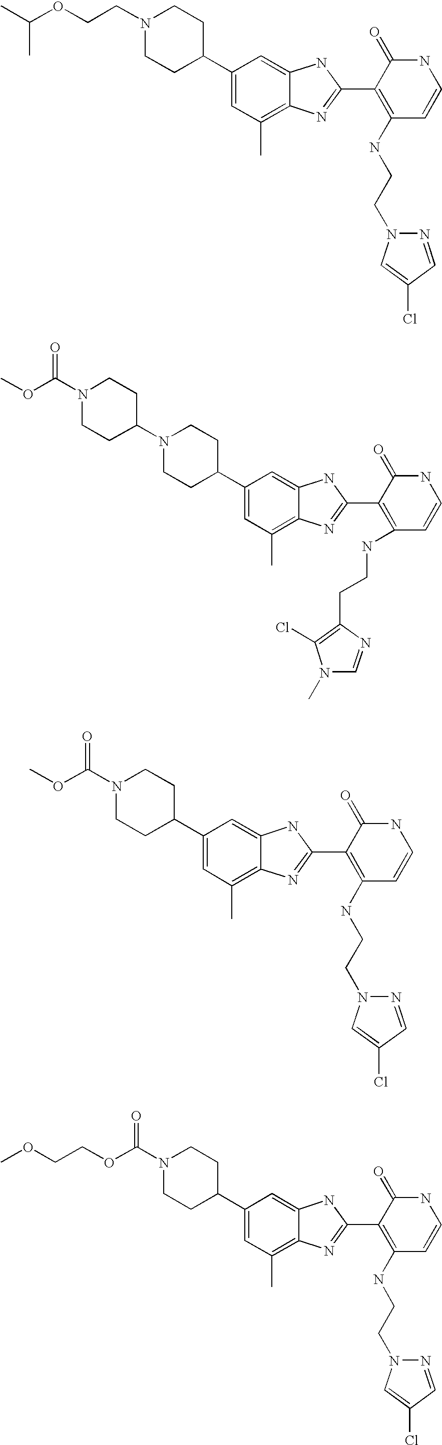

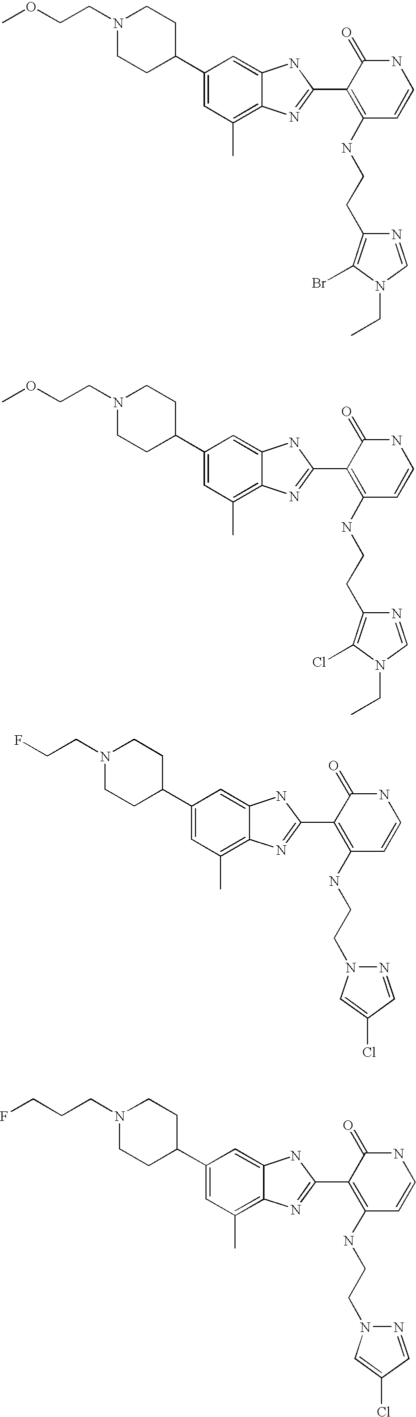

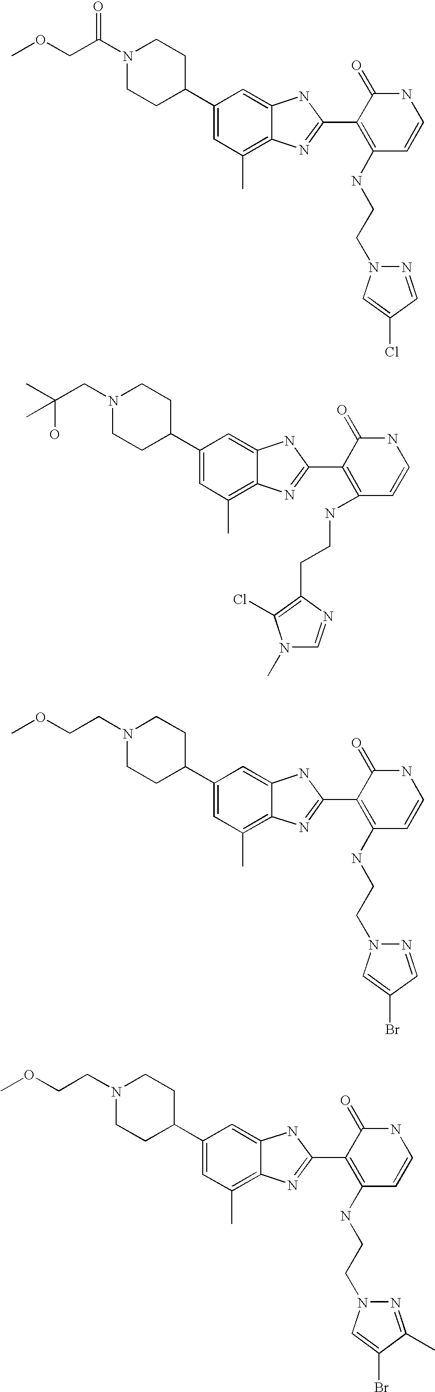

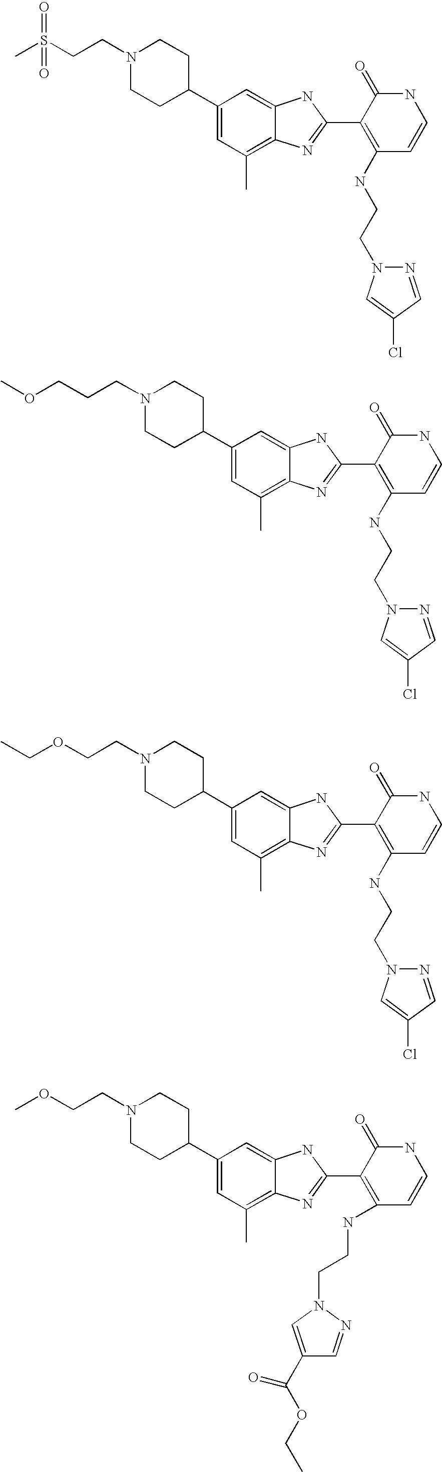

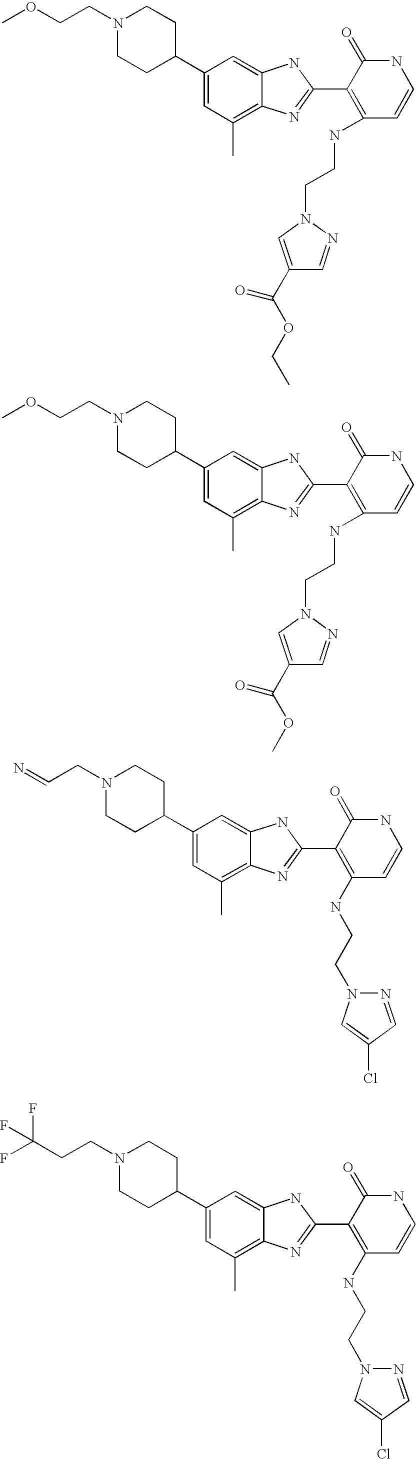

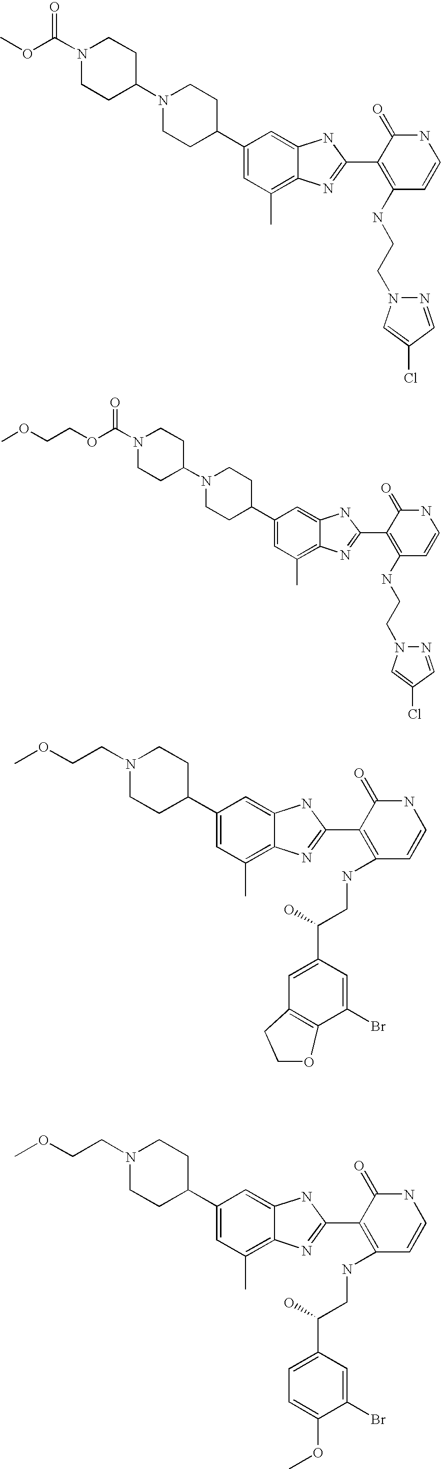

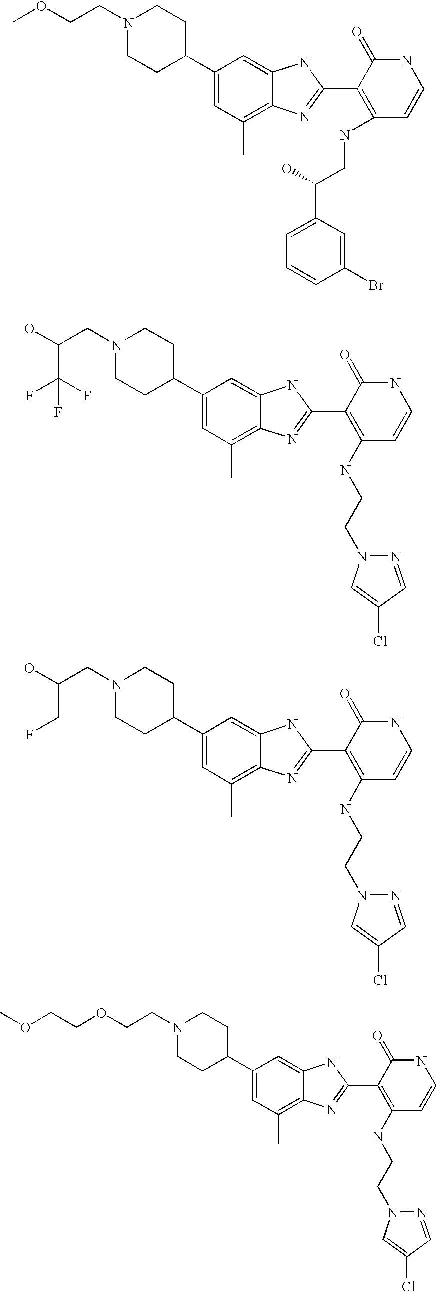

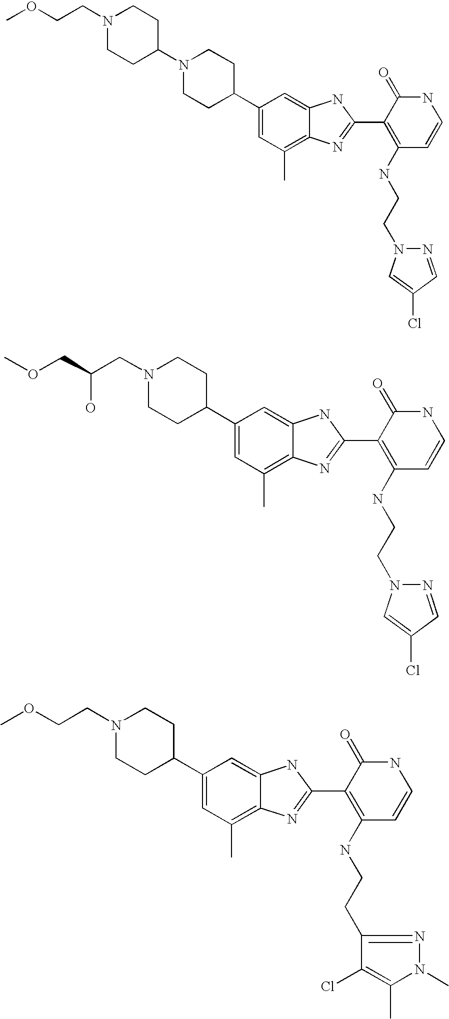

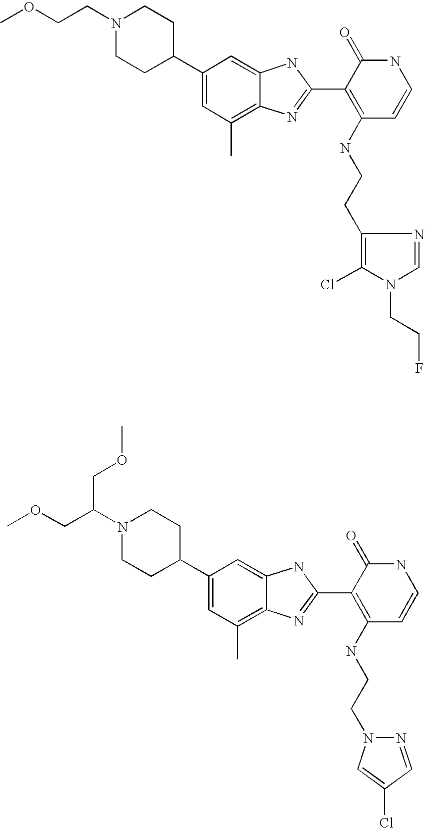

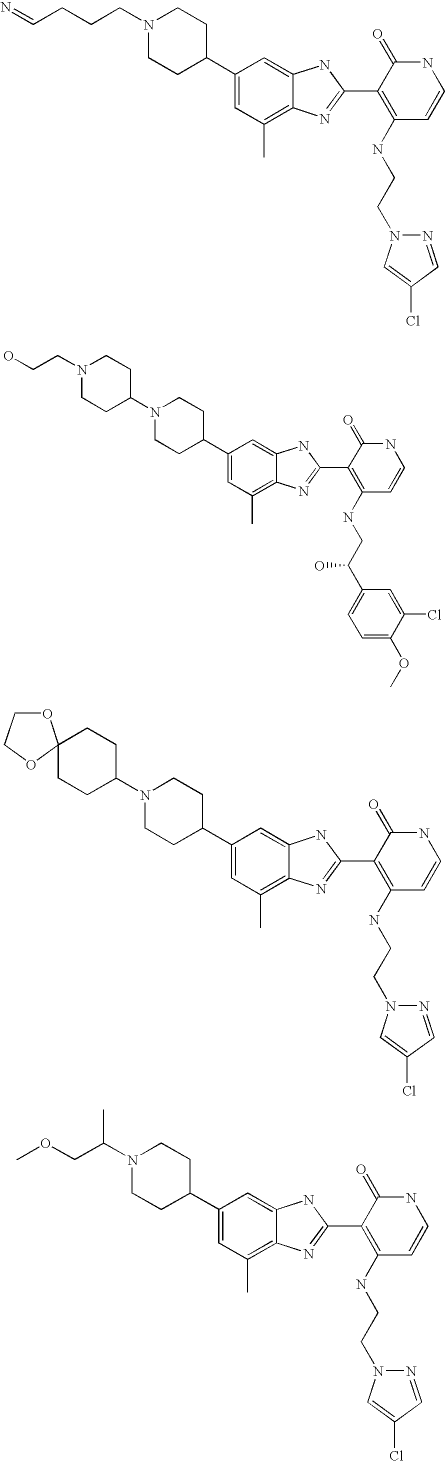

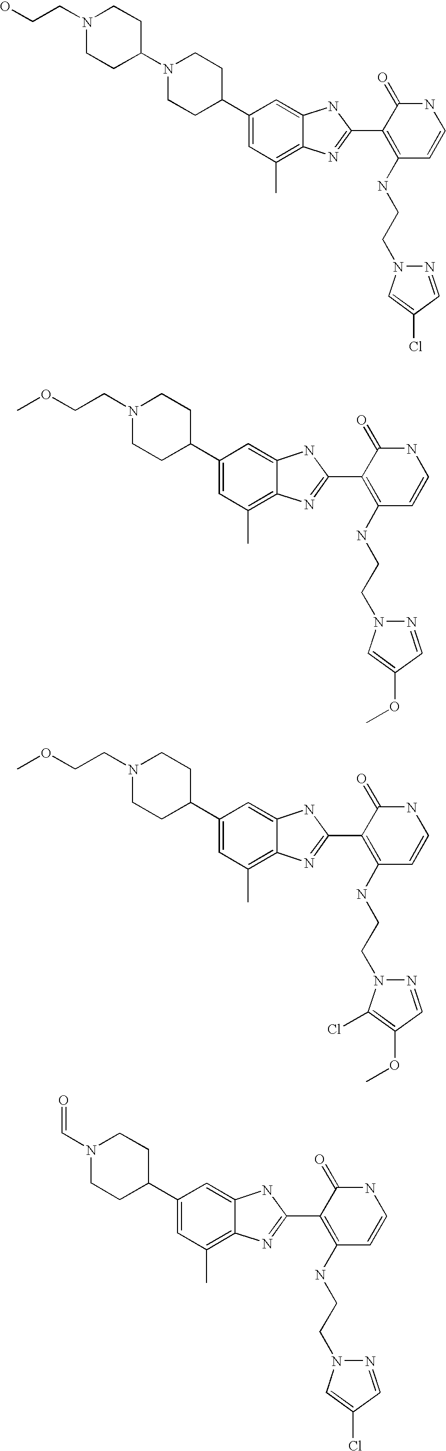

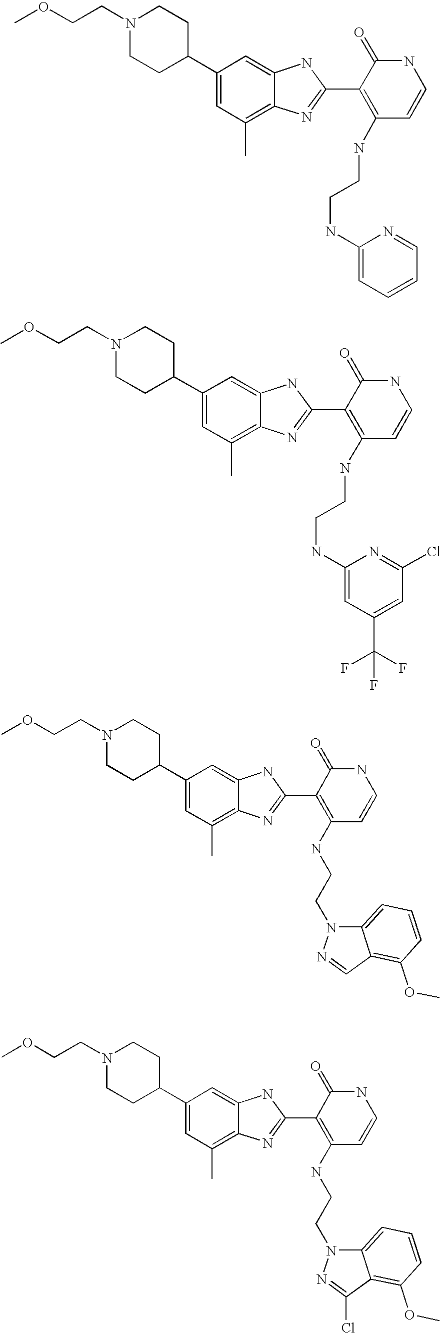

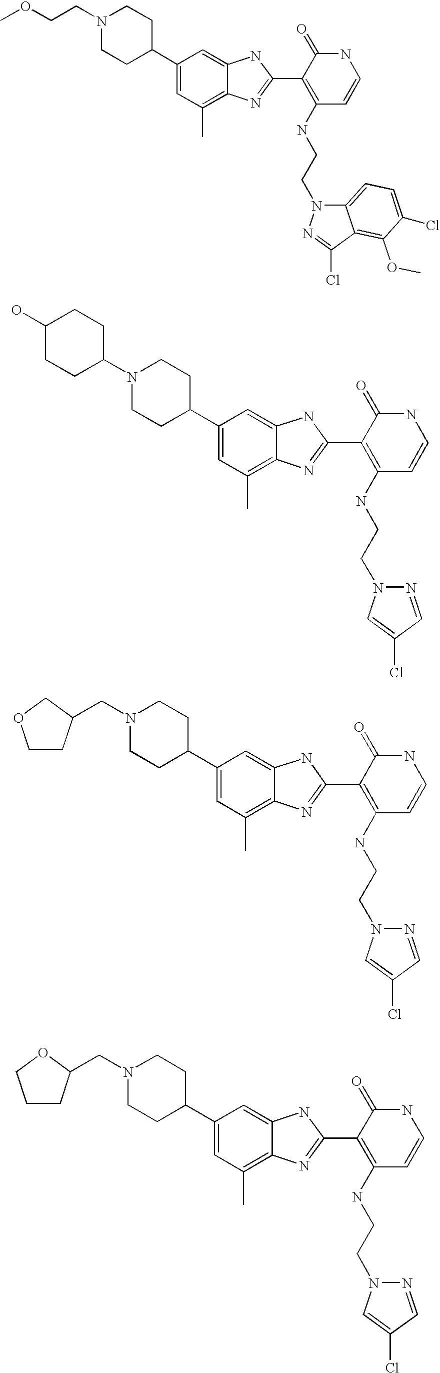

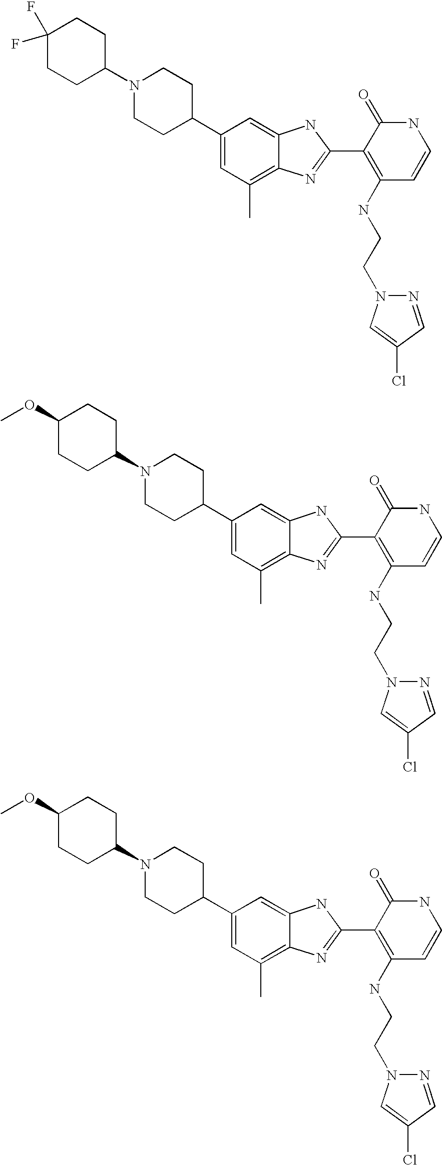

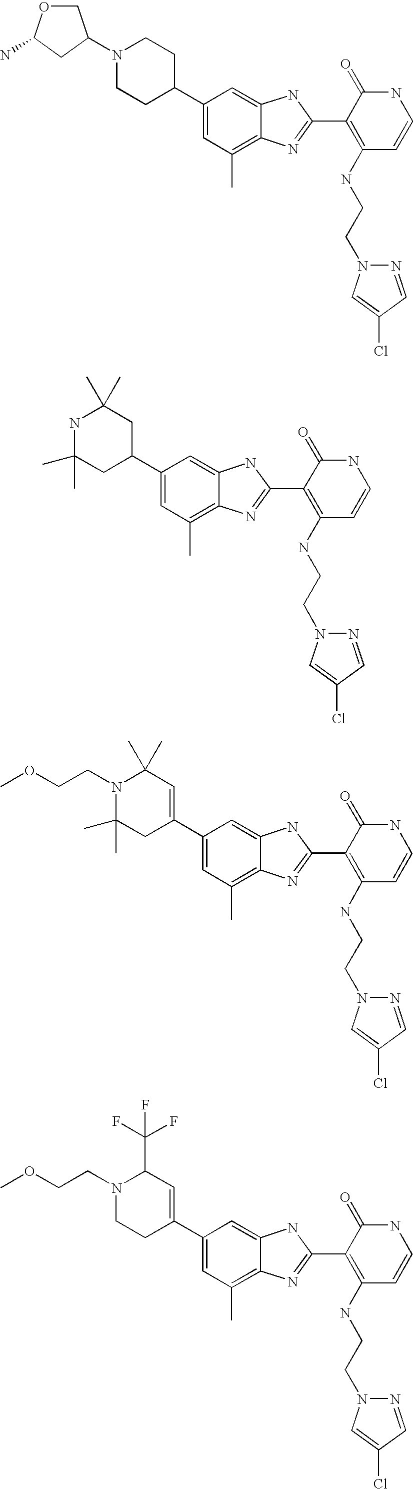

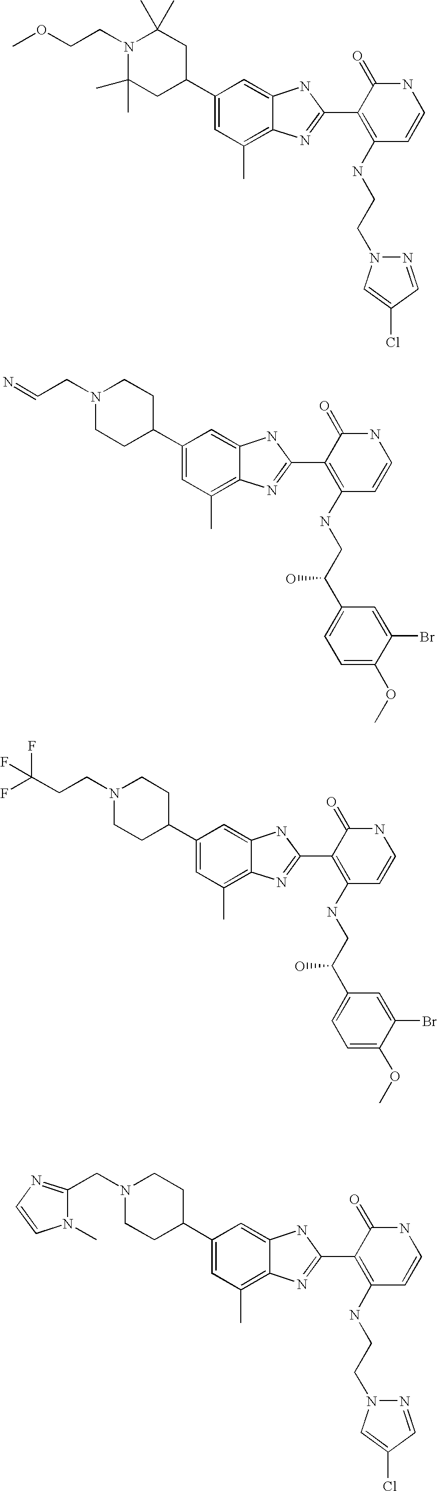

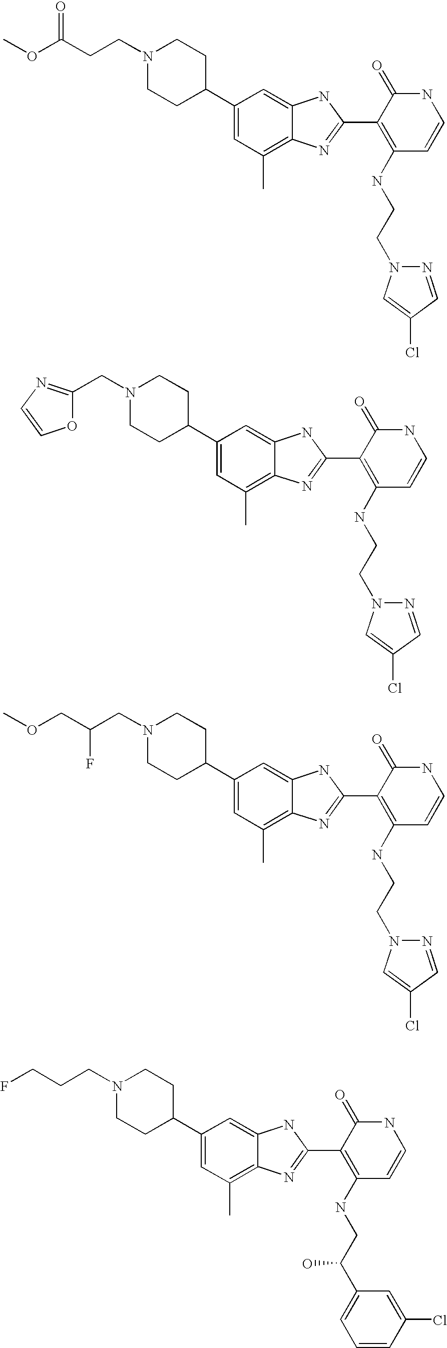

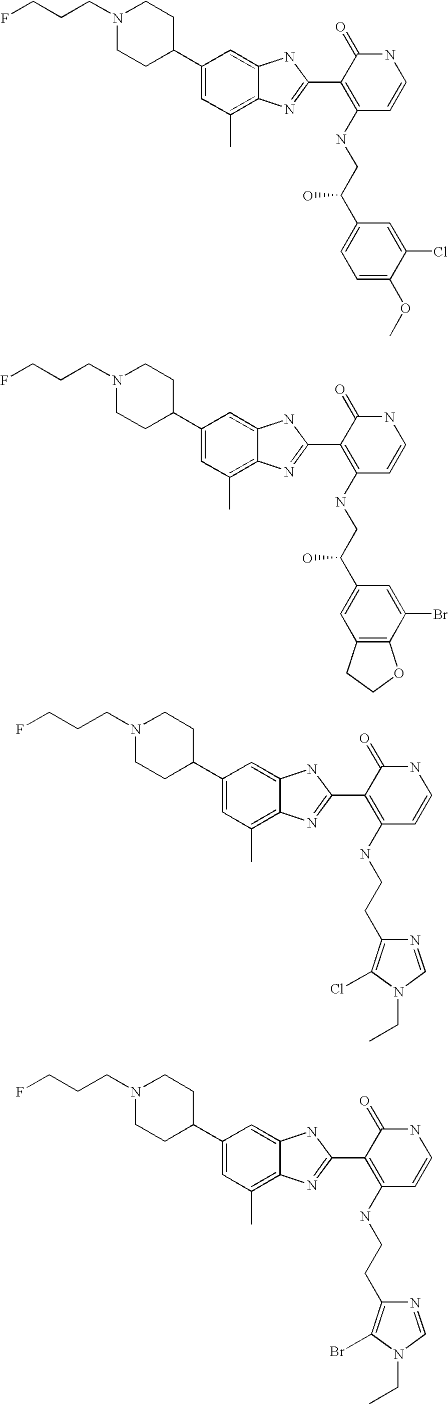

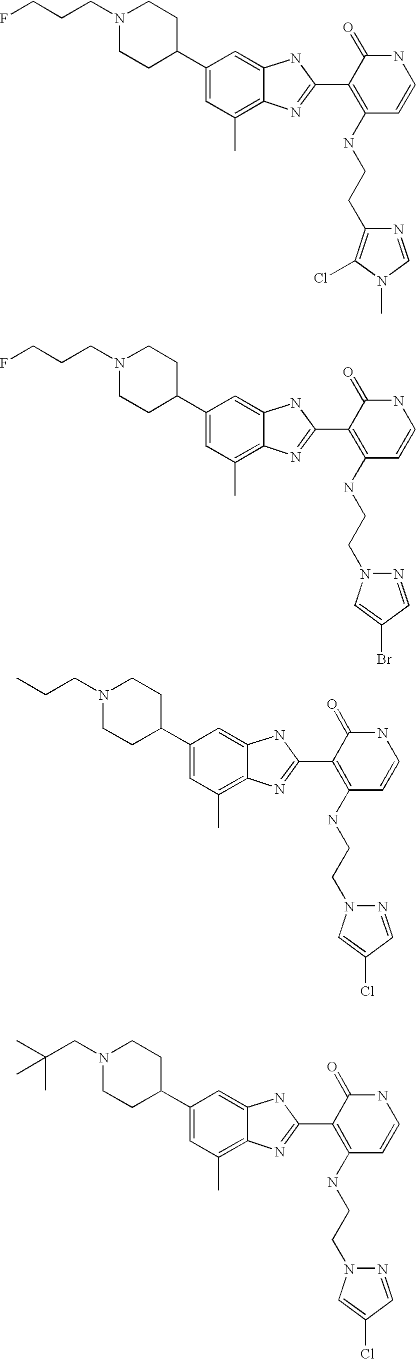

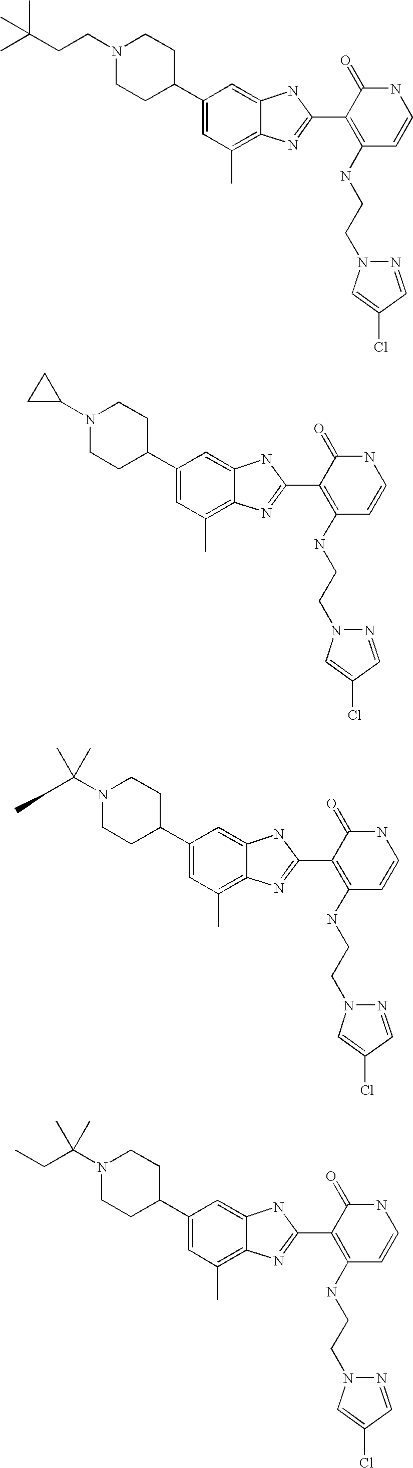

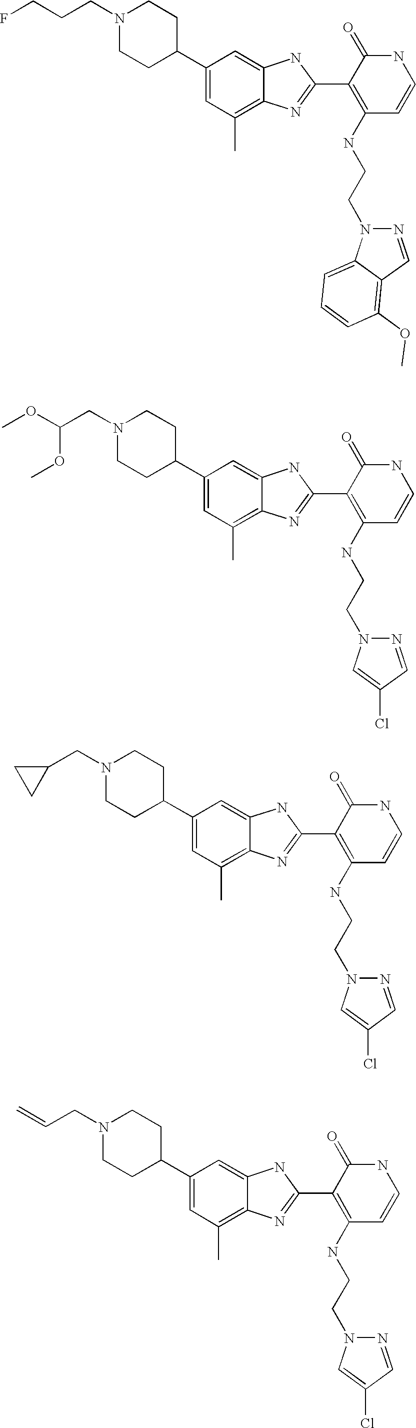

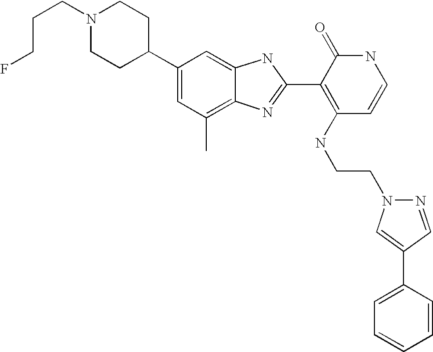

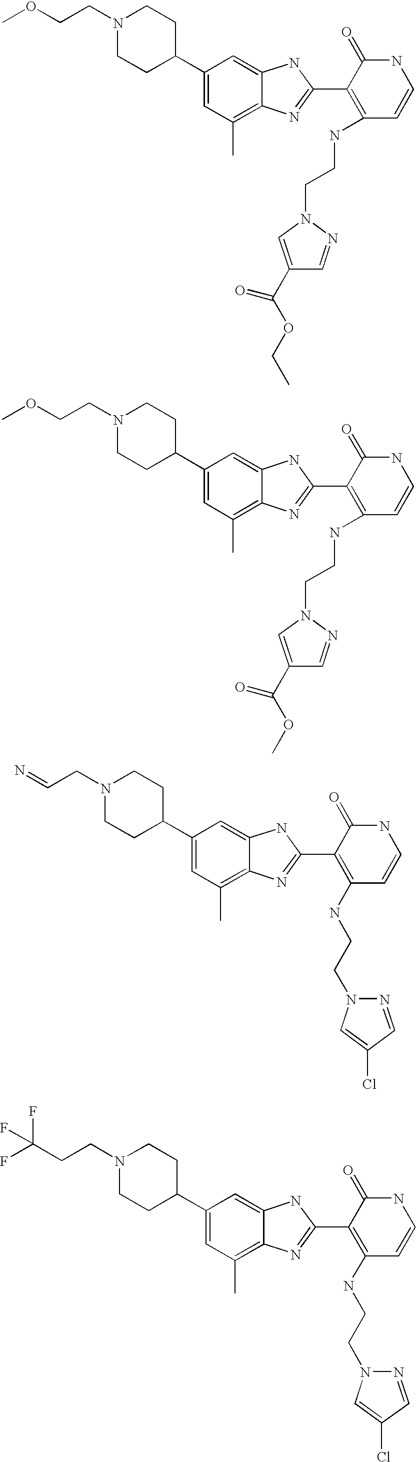

The IGF1R inhibitors of the present invention are useful in various pharmaceutically acceptable salt forms. The term “pharmaceutically acceptable salt” refers to those salt forms which would be apparent to the pharmaceutical chemist, i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients or their pharmaceutically acceptable salts in combination with pharmaceutically acceptable carriers.

The IGF1R inhibitors of the present invention are formulated as pharmaceutical compositions. These pharmaceutical compositions may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropyl-cellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS.TM. model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

When an IGF1R inhibitor is administered into a human patient, the dosage will normally be determined by the prescribing physician with the dosage generally varying according to the age, height and weight individual patient, as well as the severity of the patient's symptoms. In determining the appropriate dosage, renal function and/or hepatic function may also be evaluated and the dosing adjusted accordingly. IGF1R inhibitors may be administered as a fixed dose, ranging from about 100 to about 1000 mg/m2.

The dosage of insulin-sensitizer can also be determined by the administering physician but will typically be given in daily dosages once or twice a day wherein the dosage ranges from 500-3000 mg/day. A preferred dosage for metformin ranges from about 1000-2500 mg/day.

### EXAMPLES

### Example 1

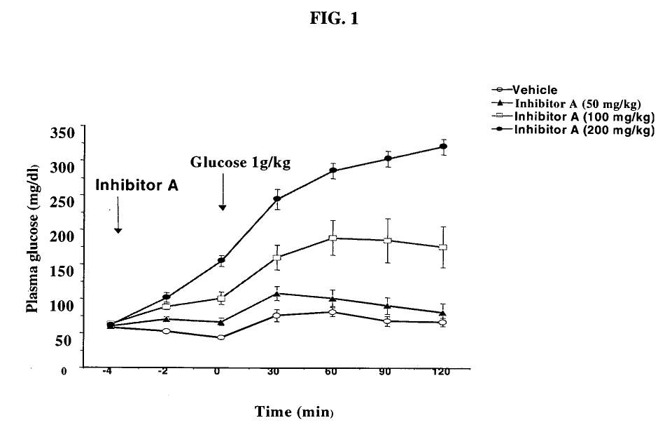

FIG. 1: The effect of inhibition of the insulin receptor was measured using an Oral Glucose Tolerance Test (OGTT). IGF1R Inhibitor A (4-[2-(3-chloro-phenyl)-2-hydroxy-ethylamino]-3-(4-methyl-6-morpholin-4-yl-1H-benzoimidazol-2-yl)-1H-pyridin-2-one) was tested in an acute OGTT study in rats: Fasted rats were orally dosed with Inhibitor A at 50, 100, and 200 mpk as a single dose for 4 hrs prior to glucose challenge (1 g/kg). Glucose was monitored at 30, 60, 90 and 120 minutes post glucose challenge using a glucometer. As is shown, there is a more pronounced effect on basal and post-glucose challenge at higher drug concentrations.

### Example 2

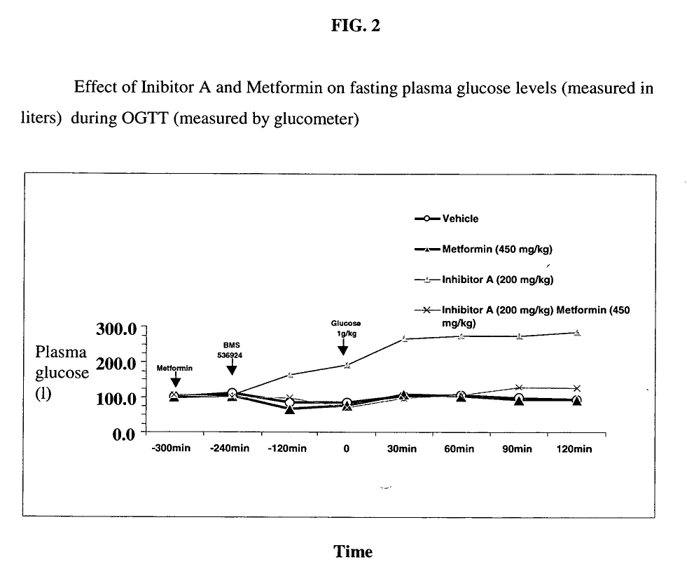

FIG. 2: An acute OGTT study to determine the effects of combination therapy using Metformin and Inhibitor A on glucose-lowering was conducted in rats. In order to stabilize glucose levels, food was withdrawn for 1 hr prior to administration of any reagent. Glucose challenge was administered 2 hrs after the administration of Inhibitor A and plasma glucose monitored. Plasma glucose levels increased upon administration of the Inhibitor (200 mpk) to 150 mg/dl prior to glucose challenge and to 250 mg/dl post-glucose challenge. However, when animals were pretreated for 1 hr with Metformin (450 mpk), the hyperglycemic effects observed were completely ameliorated indicating that Metformin counteracted Inhibitor A-induced hyperglycemic effects in rats.

The above examples are meant for illustrative purposes only and do not limit the scope of the invention in any way.

1. A method for treating a tumor that is responsive to IGF1R inhibition comprising administering to a mammal in need of such treatment an effective amount of an IGF1R inhibitor in combination with an insulin-sensitizer.

2. The method of claim 1 wherein the insulin-sensitizer is administered to said mammal prior to administration of the IGF1R inhibitor.

3. The method of claim 1 wherein said tumor is of the breast, lung, ovary, or colon.

4. The method of claim 1 wherein said insulin-sensitizer is a PPARα agonist, a PPARγ agonist or a PPAR dual agonist.

5. The method of claim 1 wherein said insulin-sensitizer is a biguanide or a glitazone.

6. The method of claim 1 wherein said insulin-sensitizer is metformin.

7. The method of claim 1 wherein said IGF1R inhibitor has the formula I: 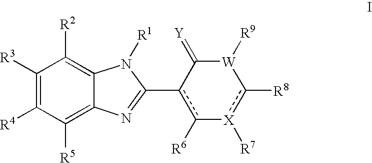
its enantiomers, diastereomers, pharmaceutically acceptable salts, hydrates, prodrugs and solvates thereof;
wherein
X is N, C or a direct bond;
Y is O or S;
W is N, C, O, or S; provided that if W is O or S, R⁹ is absent;
R¹ is H, alkyl, or alkoxy;
R² and R⁹ are independently H or alkyl;
R³ is H, C₁₋₆ alkyl, alkenyl, alkynyl, cycloalkyl, heterocycloalkyl, halo, amino, OR⁶⁰,NO₂, —OH, SR⁶⁰, —NR⁶⁰R⁶⁰, —CN, —C(O)R⁶¹, —CO₂R⁶⁰, —CONR⁶⁰R⁶⁰ OCONR⁶⁰R⁶¹, —NR⁶²CONR⁶⁰R⁶¹, —NR⁶⁰SO₂R⁶¹, —SO₂NR⁶⁰R⁶¹, —SO₂R⁶³, C(NR⁶²)NR⁶⁰R⁶¹, —C(NH⁶²)-morpholine, aryl, heteroaryl, —(CH₂)ₙC(O)₂—R⁶⁰, —NR⁶⁰R⁶¹—(CH₂)ₙOR⁶⁰, —(CH₂)ₙNR⁶⁰R⁶¹, —(CH₂)ₙSR⁶⁰, —(CH₂)ₙ aryl, —(CH₂)ₙ heteroaryl, or —(CH₂)ₙ heterocycloalkyl, wherein n is 1 to 3;
R⁴ is H, halo, alkyl or haloalkyl;
R⁵ is H, alkyl, halo, or aryl;
R⁶, R⁷, and R⁸ are each independently —NH-Z-aryl or —NH-Z-heteroaryl wherein Z is C₁-C₄ alkyl, alkenyl, or alkynyl; Z optionally having one or more hydroxy, thiol, alkoxy, thioalkoxy, amino, halo, NR⁶⁰SO₂R⁶¹ groups; Z optionally incorporating one or more groups selected from the group consisting of CO, CNOH, CNOR⁶⁰, CNNR⁶⁰, CNNCOR⁶⁰ and CNNSO₂R⁶⁰;
R⁶⁰, R⁶¹, R⁶², and R⁶³ are independently selected from the group consisting of H, alkyl, alkenyl, alkynyl, cycloalkyl, cycloalkylalkyl, hydroxy, alkoxy, aryl, heteroaryl, heteroarylalkyl, and alkyl-R²⁵;
R²⁵ is hydrogen, alkenyl, hydroxy, thiol, alkoxy, thioalkoxy, amino, alkylamino, dialkylamino, aryl, heteroaryl, cyano, halo, sulfoxy, sulfonyl, —NR³¹COOR³¹, —NR³⁰C(O)R³¹, —NR³⁰SO₂R³¹, —C(O)NR³⁰R³¹, heteroaryl or heterocycloalkyl; and
R³⁰ and R³¹ are, independently, hydrogen, alkyl, or cycloalkyl.

8. The method of claim 7 wherein W is N; X is CH; R², R⁴, R⁷ and R⁸ are H; R³ is a substituted or unsubtituted heterocycloalkyl; R⁶ is a —NH-Z-aryl or —NH-Z-heteroaryl.

9. The method of claim 8 wherein said aryl is phenyl.

10. The method of claim 8 wherein said heteroaryl is pyridinyl, imidazolyl, pyrazolyl, pyrrolyl or triazolyl.

11. The method of claim 8 wherein R³ is morpholinyl, piperazinyl, pyrrolidinyl, or piperidinyl.

12. The method of claim 1 wherein the IGF1R inhibitor is selected from the group consisting of:
(±)-4-[2-(3-Chloro-4-fluoro-phenyl)-2-hydroxy-ethylamino]-3-(6-imidazol-1-yl-4-methyl-1H-benzimidazol-2-yl)-1H-pyridin-2-one;
(S)-4-[2-(3-Fluoro-phenyl)-1-hydroxymethyl-ethylamino]-3-(6-imidazol-1-yl-4-methyl-1H-benzimidazol-2-yl)-1H-pyridin-2-one;
(±)-4-[2-(3-Chloro-phenyl)-2-hydroxy-ethylamino]-3-(6-imidazol-1-yl-1H-benzimidazol-2-yl)-1H-pyridin-2-one;
(S)-4-[2-(3-Chloro-phenyl)-2-hydroxy-ethylamino]-3-(4-methyl-6-morpholin-4-yl-1H-benzoimidazol-2-yl)-1H-pyridin-2-one;
(S)-2-[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-acetamide Bis hydrochloride;
(S)-4-[2-(3-Chloro-phenyl)-2-hydroxy-ethylamino]-3-{4-methyl-6-[4-(2-methylsulfanyl-ethyl)-piperazin-1-yl]-1H-benzoimidazol-2-yl}-1H-pyridin-2-one bis hydrochloride;
(S)₄-[2-(3-Chloro-phenyl)-2-hydroxy-ethylamino]-3-[4-methyl-6-(3R-methyl-piperazin-1-yl)-1H-benzoimidazol-2-yl]-1H-pyridin-2-one bis hydrochloride; and
(S)-4-[2-(3-Chloro-phenyl)-2-methoxy-ethylamino]-3-{6-[4-(2-hydroxy-ethyl)-piperazin-1-yl]-4-methyl-1H-benzimidazol-2-yl}-1H-pyridin-2-one.

13. The method of claim 7 wherein said IGF1R inhibitor has the formula: 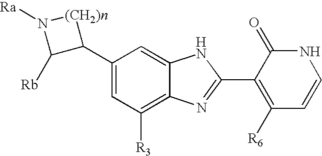
wherein
n is 0 to 4;
Rₐ is alkyl, substituted alkyl, —C(O)ₚR⁷⁰, aminolalkylamino, —SO₂R⁷¹, cycloalkyl, heterocycloalkyl, heteroaryl, or alkoxyalkoxyalkyl; wherein p is 1 or 2; R⁷⁰ and R⁷¹ are alkyl or substituted alkyl; and R_{b} is alkyl or substituted alkyl.

14. The method of claim 13 wherein Rₐ is H, cyanoalkyl, hydroxyalkyl, fluoroalkyl, methoxyalkyl, acetyl, methoxyalkylcarboxyl, dimethylaminocarbonylalkyl, methylsulfonyl, cycloalkylcarbonyl, morpholinyl, imidazolyl, isoxazolyl, or tetrahydrofuranyl.

15. The method of claim 12 wherein said insulin-sensitizer is metformin.

16. The method of claim 13 wherein said insulin-sensitizer is metformin.

17. A method of preventing IGF1R inhibitor-induced hyperglycemia in a mammal in need of such treatment comprising the administration of an insulin-sensitizer to said mammal.

18. The method of claim 17 wherein the insulin-sensitizer is metformin.

19. The method of claim 7 wherein the insulin-sensitizer is metformin and the IGF1R inhibitor is (S)-4-[2-(3-Chloro-phenyl)-2-hydroxy-ethylamino]-3-(4-methyl-6-morpholin-4-yl-1H-benzoimidazol-2-yl)-1H-pyridin-2-one.
